(19) United States
(12) Patent Application Publication
(10) Pub. No.: US 2025/0127979 A1
(43) Pub. Date: Apr. 24, 2025

(54) DEVICES, SYSTEMS, AND METHODS TO SUPPLY FLUIDS TO AN ENDOSCOPE

(71) Applicant: Boston Scientific Scimed, Inc., Maple Grove, MN (US)

(72) Inventors: Ryan V. Wales, Northborough, MA (US); Scott Edward Corbeil, Litchfield, NH (US); Ryan Vincent William Pollock, Sterling, MA (US)

(73) Assignee: Boston Scientific Scimed, Inc., Maple Grove, MN (US)

(21) Appl. No.: 18/925,661

(22) Filed: Oct. 24, 2024

Related U.S. Application Data
(60) Provisional application No. 63/592,746, filed on Oct. 24, 2023.

(51) Int. Cl. A61B 1/00; A61B 1/015

(52) U.S. Cl. CPC A61M 1/77; A61M 1/774; A61M 3/02; A61M 3/0229; A61M 3/0233; A61M 3/0254; A61B 1/00131

(57) ABSTRACT

Methods and systems for providing a flow of fluid to an endoscope. An illustrative container and tube set arranged and configured to couple to an endoscope may comprise a first container configured to contain a fluid and having a first port in fluid communication with a bottom portion thereof. A pressure vessel system may be in selective fluid communication with the first container. The pressure vessel system may include a manifold housing defining a cavity and including a first fluid inlet, a second fluid inlet, and a first fluid outlet. A first water supply tube may be in selective fluid communication with the cavity of the manifold housing and a first gas supply tube may be in operative fluid communication with the cavity of the manifold housing. A first flow control member may be positioned within the first fluid inlet.

### CROSS-REFERENCE TO RELATED APPLICATIONS

This application claims the benefit of U.S. Provisional Patent Application Ser. No. 63/592,746 filed on Oct. 24, 2023, the disclosure of which is incorporated herein by reference.

### FIELD

This disclosure relates generally to medical fluid containers and methods, and particularly to a container and tube sets to supply fluid and/or gas to an endoscope.

### BACKGROUND

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. To enable these capabilities compressed gasses from either the processor or an alternative source are used to increase the pressure within a fluid bottle which either insufflates the working lumen or washes the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day. This may introduce multiple opportunities for contamination to the tube set by either contacting non-sterile surfaces or dropping the tubing on the floor.

It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment

In a first example, a container and tube set arranged and configured to couple to an endoscope for use in an endoscopic procedure may comprise a first container configured to contain a fluid, the first container having a first port in fluid communication with a bottom portion thereof, a pressure vessel system, the pressure vessel system including a manifold housing defining a cavity and including a first fluid inlet in selective fluid communication with the first container, a second fluid inlet, and a first fluid outlet, a first water supply tube including a first end, a second end, and a first lumen extending therethrough, wherein the first lumen is in selective fluid communication with the cavity of the manifold housing and the second end of the first water supply tube is positioned external to the second container, a first gas supply tube including a first end, a second end, and a second lumen extending therethrough, wherein the second lumen is in operative fluid communication with the cavity of the manifold housing and the second end of the first gas supply tube is positioned external to the second container, and a first flow control member positioned within the first fluid inlet.

Alternatively or additionally to any of the examples above, in another example, the first flow control member may comprise a floating stopper.

Alternatively or additionally to any of the examples above, in another example, the first flow control member may comprise a one-way valve.

Alternatively or additionally to any of the examples above, in another example, the first flow control member may comprise an umbrella valve.

Alternatively or additionally to any of the examples above, in another example, the container and tube set may further comprise a second flow control member disposed between the cavity of the manifold housing and the first water supply tube.

Alternatively or additionally to any of the examples above, in another example, the second flow control member may comprise a floating stopper.

Alternatively or additionally to any of the examples above, in another example, the second flow control member may comprise a one-way valve.

Alternatively or additionally to any of the examples above, in another example, the second flow control member may comprise a linear displacement valve.

Alternatively or additionally to any of the examples above, in another example, the container and tube set may further comprise a diaphragm disposed within the cavity of the manifold housing. The diaphragm may fluidly isolate a first chamber of the cavity from a second chamber of the cavity.

Alternatively or additionally to any of the examples above, in another example, the first water supply tube may be in fluid communication with the first chamber and the first gas supply tube may be in fluid communication with the second chamber.

Alternatively or additionally to any of the examples above, in another example, the diaphragm may be formed from a flexible material.

Alternatively or additionally to any of the examples above, in another example, the container and tube set may further comprise a piston disposed within the cavity of the manifold housing. The piston may have a seal member disposed around a perimeter thereof and fluidly isolate a first chamber of the cavity from a second chamber of the cavity.

Alternatively or additionally to any of the examples above, in another example, the container and tube set may further comprise a biasing member disposed between an end of the piston and the first fluid outlet. The biasing member may be configured to bias the piston towards the second fluid inlet.

Alternatively or additionally to any of the examples above, in another example, the first fluid inlet may comprise a spike port adaptor.

Alternatively or additionally to any of the examples above, in another example, the container and tube set may further comprise a second water supply tube including a first end, a second end, and a third lumen extending therethrough. The third lumen may be in selective fluid communication with the cavity of the manifold housing via a second fluid outlet and the second end of the second water supply tube may be positioned external to the second container.

In another example, a container and tube set arranged and configured to couple to an endoscope for use in an endoscopic procedure may comprise a first container configured to contain a fluid, the first container having a first port in fluid communication with a bottom portion thereof and a pressure vessel system. The pressure vessel system may comprise a tubing valve including a central lumen extending from a proximal end to a distal end thereof and one or more channels laterally spaced from the central lumen and extending from the proximal end to the distal end of the tubing valve, a proximal gas supply tubing including a proximal end, a distal end coupled to the proximal end of the tubing valve, and a first lumen extending therethrough, wherein the first lumen is in fluid communication with the one or more channels of the tubing valve, a proximal water supply tubing including a proximal end, a distal end coupled to the proximal end of the tubing valve, and a second lumen extending therethrough, wherein the second lumen is in selective fluid communication with the central lumen of the tubing valve and the first container, a distal gas supply tubing including a proximal end coupled to the distal end of the tubing valve, a distal end, and a third lumen extending therethrough, wherein the third lumen is in fluid communication with the one or more channels of the tubing valve, and a distal water supply tubing including a proximal end coupled to the distal end of the tubing valve, a distal end, and a fourth lumen extending therethrough, wherein the second lumen is in selective fluid communication with the central lumen of the tubing valve.

Alternatively or additionally to any of the examples above, in another example, the pressure vessel may further comprise a slit valve disposed between the distal end of the proximal water supply tubing and the proximal end of the distal water supply tubing.

Alternatively or additionally to any of the examples above, in another example, the pressure vessel may further comprise a push button valve assembly disposed in-line with the proximal water supply tube.

Alternatively or additionally to any of the examples above, in another example, the container and tube set may further comprise a one-way valve disposed in a wall of the proximal water supply tubing.

In another example, a drive system for generating torque to supply a fluid to an endoscope system may comprise a tubular housing including a first end, a second end and a lumen extending therethrough, the tubular housing including a first air inlet and a first air outlet disposed adjacent to the first end thereof and a turbine and drive unit assembly rotatably disposed within the lumen of the tubular housing. The turbine and drive assembly may comprise a turbine, a drive unit, and an elongate shaft extending between the turbine and the drive unit. Rotation of the turbine may be configured to rotate the drive unit.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3 depicts another illustrative endoscope system having an alternative fluid supply system;

FIG. 4 is a side view of an alternative pressure vessel system;

FIG. 5A is a partial cross-sectional view of the pressure vessel system of FIG. 4 in a first configuration, taken at line 5A-5A of FIG. 4;

FIG. 5B is a partial cross-sectional view of the pressure vessel system of FIG. 4 in a second configuration;

FIG. 6 is a proximal perspective view of an illustrative tubing valve;

FIG. 7 is a cross-sectional view of another illustrative pressure vessel system;

FIG. 8A is a cross-sectional view of another illustrative pressure vessel system in a first configuration;

FIG. 8B is a cross-sectional view of the illustrative pressure vessel system of FIG. 8A in a second configuration;

FIG. 9 is a cross-sectional view of another illustrative pressure vessel system in a first configuration;

FIG. 10A is a schematic cross-sectional view of another illustrative pressure vessel system in a first configuration;

FIG. 10B is a schematic cross-sectional view of the illustrative system of FIG. 10A in a second configuration;

FIG. 11A is a cross-sectional view of another illustrative pressure vessel system in a first configuration;

FIG. 11B is a cross-sectional view of the illustrative system of FIG. 10A in a second configuration;

FIG. 12A is a perspective view of a first end of an illustrative drive system for generating torque to supply a lens wash fluid;

FIG. 12B is a perspective view of a second end of the system of FIG. 12A.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. To enable these capabilities compressed gasses from either the processor or an alternative source are used to increase the pressure within a fluid bottle which either insufflates the working lumen or washes the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day which may introduce multiple opportunities for contamination to the tube set by either contacting non-sterile surfaces or dropping the tubing on the floor. Additionally, current water bottle and tube sets may leak if not threaded properly. Finally, the water bottle may require a level surface to be placed properly which, in an endoscopy suite, may be at a premium. Disclosed herein are methods and systems to reduce or eliminate the need to disconnect the tube set and use a second bottle. In some cases, the methods and systems disclosed herein may enable lens wash without the use of a secondary fluid vessel.

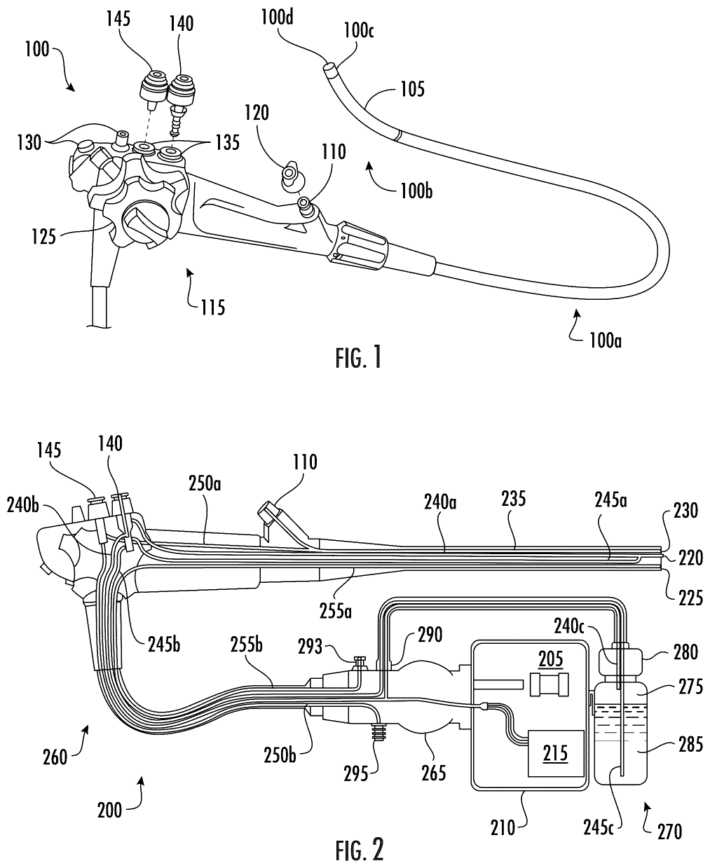

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connector portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top of the water reservoir. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

It is contemplated that other arrangements for the fluid sources may be used as desired. For example, in some cases, water for irrigation and lens wash may come from a same container. Some illustrative systems and method to supply fluids to the endoscope are described in commonly assigned U.S. Patent Application No. 63/419,900, titled DEVICES, SYSTEMS, AND METHODS TO SUPPLY FLUIDS TO AN ENDOSCOPE, the disclosure of which is hereby incorporated by reference.

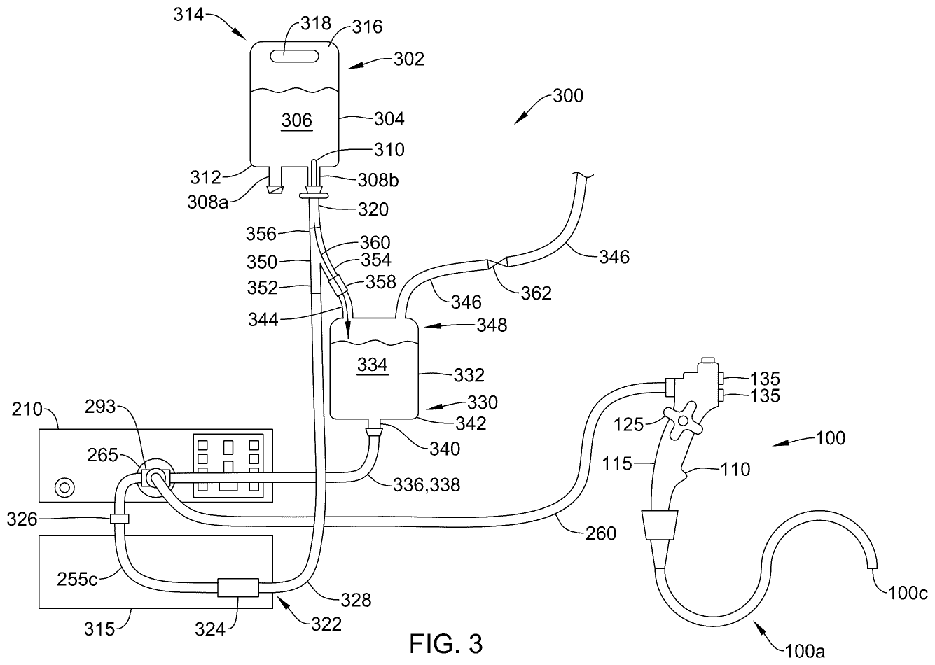

FIG. 3 depicts a schematic view of another illustrative endoscopic system 300 which may reduce the number of water reservoir changes and/or reduce opportunities for contamination during replacement of the water reservoir(s). The system 300 may include a number of advantages over the current bottle system described above. The system 300 may include components similar to the endoscope and endoscope systems described with regard to FIGS. 1-2; however, not all features may be described or shown here.

Generally, the system 300 may include a first reservoir 302 and a second reservoir 330. The first reservoir 302 may be configured to supply water or fluid for both irrigation (e.g., via the first reservoir 302) and lens wash (e.g., via the second reservoir 330). This may allow a single fluid source to be used to provide fluid for both irrigation and lens wash. While not explicitly shown, the reservoirs 302, 330 may include printed lines, numbers, or other visual indicia to allow a user to easily determine how much fluid is left in the reservoirs 302, 330.

The first reservoir 302 may include a first container 304 configured to hold a first volume of fluid 306. In the illustrated embodiment, the first container 304 is fluidly coupled to the upstream irrigation supply tubing 328 and is configured to provide fluid for irrigation to the endoscope 100. Generally, the irrigation supply tubing 328 may be a water or fluid supply line or tube for supplying water or other fluid to an endoscope. Additionally, the first container 304 may be selectively fluidly coupled to a second fluid reservoir 330. The second reservoir 330 may include a second container 332 configured to hold a second volume of fluid 334. In the illustrated embodiment, the second container 332 is fluidly coupled to the gas and lens wash supply tubing 336, 338 and is configured to provide fluid for lens wash to the endoscope 100. Generally, the lens wash supply tubing 338 may be a water or fluid supply line or tube for supplying water or other fluid to an endoscope. The gas and lens wash supply tubing 336, 338 may be coaxially arranged. For example, the gas supply tubing 336 may define a lumen that is sufficiently large in diameter to encompass a smaller diameter lens wash tubing 338, coaxially received within the gas supply tubing 336, as well as provide air to the water source in an annular space surrounding the lens wash tubing 338 to pressurize the second reservoir 330. The lens wash supply tubing 338 may be configured to exit the lumen defined by the coaxial gas supply tubing 336 in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265. In other embodiments, the gas and lens wash supply tubing 336, 338 may be arranged in a side-by-side arrangement.

The first and second containers 304, 332 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first and second containers 304, 332 may be entirely translucent, entirely opaque, or combinations thereof. In some cases, the first and second containers 304, 332 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first and second containers 304, 332 may be variable. For example, the volume of the first container 304 and/or the second container 332 may be 500 milliliters (mL) or greater, 1000 mL or greater, 2000 mL or greater, 3000 mL, 4000 mL or greater, etc. The volume may be less than 500 mL or greater than 4000 mL, as desired. One or both of the first and second reservoirs 302, 330 may be pre-filled (e.g., prior to entering the procedure suite or at the time of manufacturing) with water or other fluid. In some cases, the clinician may select the reservoir(s) 302, 330 from a plurality of differently sized available reservoirs based on the number and/or types of procedures expected for a typical or the specific day. In the illustrated embodiments, the first reservoir 302 may supply fluid to the second reservoir 330. By selecting a first reservoir 302 having a volume large enough to accommodate an entire day of procedures, the need for replacing the sterile fluid source (e.g., the first reservoir 302) may be reduced or eliminated. In some cases, the first reservoir 302 may be used to periodically refill the second reservoir 330. Thus, the volume of the first reservoir 302 may be greater than the volume of the second reservoir 330, although this is not required. It is further contemplated that, in some embodiments, one or both of the first or second reservoirs 302, 330 may be a rigid bottle.

It is contemplated that flexible bags may utilize less plastic (or other material) than a bottle designed to hold a similar amount of fluid. Thus, the use of a flexible bag as a fluid reservoir 302, 330 may increase the level of environmental sustainability of the system 300. For example, if the user sets up the system with a 3000 mL (3 liter) bag reservoir 302 and therefore does not need to utilize three individual one liter bottles, a significant reduction of waste may be realized. It is further contemplated that when disposed of or discarded, a flexible bag reservoir may occupy less volume than a bottle capable of holding an equivalent amount of fluid.

The first reservoir 302 may further include one or more ports 308a, 308b, such as, but not limited to a spike port or a septum port, extending from and in selective fluid communication with an interior of the first container 304. The ports 308a, 308b may be formed as a monolithic structure with the first container 304. The ports 308a, 308b may be generally tubular structures with each port 308a, 308b defining a lumen extending therethrough. The lumens of the ports 308a, 308b may be configured to selectively fluidly couple the interior of the first container 304 with another component, such as, but not limited to, a fluid or water supply tube. In some embodiments, the ports 308a, 308b may be positioned adjacent to a bottom end 312 of the first reservoir 302. However, this is not required. The ports 308a, 308b may be positioned in other locations, as desired. If the ports 308a, 308b are positioned at a location other than the bottom end 312 of the first container 304, a dip tube or tube extension may be required to access the fluid at the bottom of the first container 304. In some cases, at least one port 308b may be configured to be coupled to the upstream irrigation tubing (or water supply tube) 328 while another port 308a may be configured to allow the user to add additives to the fluid 306. In other examples, the upstream irrigation tubing 328 may be coupled to the first port 308a while the second reservoir 330 is in fluid communication with the second port 308b. While the first reservoir 302 is illustrated as including two ports 308a, 308b, the first reservoir 302 may include one port or more than two ports, as desired.

While not explicitly shown, the ports 308a, 308b may each include a removable cap or seal configured to form a fluid tight seal with the port 308a, 308b. The removable cap or seal may help to maintain the sterility of the ports 308a, 308b. The removable cap or seal may be coupled to a free end of the ports 308a, 308b using a number of different techniques. For example, the cap or seal may be coupled to the port 308a, 308b using a threaded engagement, a friction fit, a snap fit, etc. In other instances, the cap or seal may be removed through a twisting motion configured to break the cap or seal from the port 308a, 308b. Once the cap or seal has been removed, the port 308a, 308b may be pierced with a spike tip or spike port adaptor 310 that is coupled to the upstream irrigation tubing 328. For example, in addition to the removable cap or seal, the port 308a, 308b may include an internal seal disposed within a lumen of the port 308a, 308b that may be punctured or pierced by the spike port adaptor 310. The internal seal may be configured to prevent fluid 306 from leaking from the first container 304 prior to the spike port adaptor 310 being inserted into the port 308a, 308b. In some embodiments, the internal seal may be self-sealing such that upon removal of the spike port adaptor 310 fluid is prevented from leaking from the port 308a, 308b. The outer surface of the spike port adaptor 310 may form an interference fit with the inner surface of the port 308a, 308b. The fit and/or coupling between the spike port adaptor 310 and the port 308a, 308b may be sufficient to remain in place when the irrigation supply tube 328, branched connector 350, and/or other tubing sets are coupled to the spike port adaptor 310. It is contemplated that the spike port adaptor 310 may be inserted into one of the ports 308a, 308b utilizing universally used aseptic techniques such as those used with IV fluid bags. This may help reduce infection risk by maintaining sterile components, not introducing contaminants into the fluid 306, etc. It is further contemplated that additives may be added to the fluid 306 using similar aseptic techniques via one of the ports 308a, 308b.

The first reservoir 302 may include a handle 316 positioned adjacent to a top portion 314 thereof. The handle 316 may define an opening or through hole 318 for receiving a hand or hook therethrough to carry the first reservoir 302. In some cases, the handle 316 may include an undulating surface configured to provide a more ergonomic grip for the user. It is contemplated that the handle 316 may be formed from a similar material as the first container 304 or a different material, as desired. In some examples, the handle 316 may be formed from polyethylene terephthalate (PET), polypropylene (PP), etc. The handle 316 may allow the first reservoir 302 to be hung from a hook, such as, but not limited to an IV stand. Hanging the first reservoir 302 may allow the first reservoir 302 to be positioned above the level of an endoscope cart which may enable the user to see the fluid 306 level at any time. This may help the clinician avoid running out of fluid during a procedure. Additionally, elevating the reservoir may eliminate the need for the clinician to bend or stoop during setup of the system 300 and/or to change the first reservoir 302. In some cases, head pressure generated from elevating the first reservoir 302 may enable rapid priming of the irrigation circuit (and/or lens wash circuit if so connected) which may save time during setup. It is further contemplated that hanging the first reservoir 302 from a hook or IV stand may allow the first reservoir 302 to be positioned away from expensive capital equipment thus reducing or eliminating the potential for fluid running or flowing inadvertently onto the capital equipment and causing damage or destruction.

The first reservoir 302 may be connected in fluid communication with a lumen of the upstream irrigation supply tube 328. The upstream irrigation supply tube 328 extends from a second end region 322 external to the container 304 and positioned within a pump head 324 of the peristaltic irrigation pump 315 to a first end 320. The first end 320 of the upstream irrigation supply tube 328 is coupled to the spike port adaptor 310 which in turn is configured to extend through a lumen of the port 308b and pierce a seal within the lumen of the port 308b to fluidly couple the interior of the container 304 with the lumen of the upstream irrigation supply tube 328. The second end of the upstream irrigation supply tube 328 is configured to be fluidly coupled with an irrigation lumen of the endoscope 100. When irrigation water is required, fluid is pumped from the first container 304 by operating the irrigation pump 315, such as by depressing a footswitch (not shown), and flows from the first reservoir 302, through the upstream irrigation supply tubing 328 and a branched connector 350, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in line with the downstream irrigation supply tubing 255c. The flow control valve 326 may prevent the unintentional flow of fluid from the first container 304 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the first reservoir 302 and the irrigation pump 315. The flow control valve 326 may also prevent fluid from leaking from the downstream irrigation supply tube 255c when the endoscope 100 is changed between patients and the tubing set connector is separated from the endoscope water port.

In some embodiments, the irrigation pump 315 may be omitted. For example, the reservoir 302 may be inserted into a compression sleeve. When irrigation fluid is desired, the compression sleeve may be activated to exert pressure on an outer surface of the reservoir 302 and to provide the required pressure to perform irrigation at the distal end of the endoscope 100. In another embodiment the reservoir 302 may be inserted into a compression sleeve which applies constant pressure to the reservoir 302 with a flow switch positioned along irrigation supply tubing 328 to provide binary control of irrigation flow.

The second reservoir 330 may further include one or more ports 340, such as, but not limited to a spike port or a septum port, extending from and in selective fluid communication with an interior of the second container 332. The port 340 may be formed as a monolithic structure with the second container 332. The port 340 may be a generally tubular structure with the port 340 defining a lumen extending therethrough. The lumen of the port 340 may be configured to selectively fluidly couple the interior of the second container 332 with another component, such as, but not limited to, fluid/water/gas supply tube(s). In some cases, the port 340 may be configured to be coupled to the gas and lens wash supply tubing 336, 338. In some embodiments, the port 340 may be positioned adjacent to a bottom end 342 of the second reservoir 330. However, this is not required. The port 340 may be positioned in other locations, as desired. If the port 340 is positioned at a location other than the bottom end 342 of the second container 332, a dip tube or tube extension may be required (e.g., coupled to the lens wash supply tubing 338) to access the fluid at the bottom of the second container 332. While the second reservoir 330 is illustrated as including one port 340, the second reservoir 330 may include more than one port, as desired.

While not explicitly shown, the port 340 may include a removable cap or seal configured to form a fluid tight seal with the port 340. The removable cap or seal may help to maintain the sterility of the port 340. The removable cap or seal may be coupled to a free end of the port 340 using a number of different techniques. For example, the cap or seal may be coupled to the port 340 using a threaded engagement, a friction fit, a snap fit, etc., or may be fixedly coupled using a number of techniques such as adhesive or solvent bonding. In other instances, the cap or seal may be removed through a twisting motion configured to break the cap or seal from the port 340. Once the cap or seal has been removed, the port 340 may be pierced with a spike tip or spike port adaptor (not explicitly shown) that is coupled to the gas and lens wash supply tubing 336, 338. For example, in addition to the removable cap or seal, the port 340 may include an internal seal disposed within a lumen of the port 340 that may be punctured or pierced by the spike port adaptor. The internal seal may be configured to prevent fluid 334 from leaking from the second container 332 prior to the spike port adaptor being inserted into the port 340. In some embodiments, the internal seal may be self-sealing such that upon removal of the spike port adaptor fluid is prevented from leaking from the port 340. The outer surface of the spike port adaptor may form an interference fit with the inner surface of the port 340. The fit and/or coupling between the spike port adaptor and the port 340 may be sufficient to remain in place when the gas and fluid supply tubing 336, 338 and/or other tubing sets are coupled to the spike port adaptor 310. It is contemplated that the spike port adaptor may be inserted into one of the ports 340 utilizing universally used aseptic techniques such as those used with IV fluid bags. This may help reduce infection risk by maintaining sterile components, not introducing contaminants into the fluid 334, etc. It is further contemplated that additives may be added to the fluid 334 using similar aseptic techniques via one of the ports 340, if so desired. In some cases, other coupling mechanisms may be used as desired to couple the gas and lens wash supply tubing 336, 338 to the port 340. Some illustrative coupling mechanisms may include, but are not limited to, threaded engagements, snap fits, friction fits, quick connect style couplers, etc., or may be fixedly coupled using a number of techniques such as adhesive or solvent bonding.

The gas supply tubing 336 extends from a second end external to the second container 332 to the port 340. The gas supply tubing 336 may extend into the interior of the second container 332 and terminate within a reservoir gap (e.g., above the level of the fluid 334). However, in some cases, the gas supply tubing 336 may terminate within the fluid 334. A lumen extends through the gas supply tubing 336 for receiving a flow of air and/or gas therethrough. The lumen of the gas supply tubing 336 may be in operative fluid communication with a top portion of the interior of the second container 332. The lens wash supply tubing 338 extends from a second end external to the second reservoir 330 to a first end in fluid communication with a bottom portion 342 of the second container 332. In some embodiments, the lens wash supply tubing 338 may terminate at the port 340. A lumen extends through the lens wash supply tubing 338 for receiving a flow of fluid therethrough. The lumen of the lens wash supply 338 is in selective operative fluid communication with a bottom portion 342 of the second container 332. In the illustrated embodiment, the gas supply tubing 336 and the lens wash supply tubing 338 may couple to the second container 332 through a single or common opening (e.g., port 340). For example, the gas supply tubing 336 and the lens wash supply tubing 338 may be coaxially arranged. However, this is not required. In some cases, the gas supply tubing 336 and the lens wash supply tubing 338 may extend in a side by side arrangement or may be separately connected to the second container 332 in different locations.

The second container 332 may further include a first fluid inlet 344 and a second fluid inlet 346. While the first and second fluid inlets 344, 346 are illustrated as being adjacent to or extending from a top portion 348 of the second container 332, the first and/or second fluid inlets 344, 346 may be positioned at other locations about the second container 332, as desired. In some embodiments, the first and/or second fluid inlets 344, 346 may be tubular members formed as a single monolithic structure with the second container 332. In other embodiments, the first and/or second fluid inlets 344, 346 may include tubular components releasably coupled to ports (similar in form and function to port 340) formed in or with the container 332.

The first fluid inlet 344 may be in selective fluid communication with the first reservoir 302. For example, a branched connector 350 may be positioned in-line with the upstream irrigation tubing 328. In some embodiments, the branched connector 350 may be a “Y” connector or a “T” connector having an inlet leg 356 defining a first fluid inlet, a first outlet leg 352 defining a first fluid outlet, and a second outlet leg 354 defining a second fluid outlet. However, it is contemplated that the branched connector 350 may include more than one fluid inlet and fewer than two or more than two fluid outlets, if so desired.

The branched connector 350 may be positioned in-line with the upstream irrigation tubing 328 such that the inlet leg 356 and the first outlet leg 352 are fluidly coupled with the lumen of the upstream irrigation tubing 328. Fluid may flow from the first reservoir 302, through the upstream irrigation tubing 328, through the branched connector 350 and again through the upstream irrigation tubing 328. The branched connector 350 may be positioned such that the inlet leg 356 is upstream of the outlet legs 352, 354 relative to a flow of irrigation fluid. In some embodiments, the branched connector 350 and the spike port 310 may be molded or formed as a single monolithic structure. It is contemplated that this may reduce connection points in the fluid circuit. In such an instance, the first end 320 of the irrigation supply tubing 328 may be fluidly coupled to the first outlet leg 352 of the branched connector 350.

The second outlet leg 354 may be fluidly coupled to the first fluid inlet 344 of the second reservoir 330. A flow control mechanism, such as, but not limited to, a one-way valve 358 may be positioned between the second fluid outlet of the second outlet leg 354 and the first fluid inlet 344 of the second reservoir 330 to selectively fluidly couple the second container 332 with the first container 304. The one-way valve 358 may be configured to be opened to allow fluid to selectively pass from the first reservoir 302 to the second reservoir 330 while preventing fluid (e.g., gas, water, or other fluid) from exiting the second container 332 and entering the irrigation supply tubing 328 and/or the first container 304. In some embodiments, the one-way valve 358 may be replaced with a clamp which may compress the first fluid inlet 344 to selectively fluidly isolate the second container 332 from the first container 304 and removed to selectively couple the second container 332 with the first container 304. In yet other embodiments, the one-way valve 358 may be replaced with a spring-loaded valve, a stopcock, or other two-way valve. When it is desired to add fluid to the second reservoir 330 from the first reservoir 302, the one-way valve 358 (or other flow control mechanism) may be opened or released. Fluid may then be at least partially diverted from the irrigation supply tubing 328 through the second outlet leg 354 of the branched connector 350 and into the second container 332 along flow path 360. Fluid may be added to the second container 332 while the irrigation pump 315 is running or while the irrigation pump 315 is idle, as desired.

The second fluid inlet (or gas supply tube) 346 of the second container 332 may be an alternative gas supply tubing configured to be coupled to an alternative gas supply (e.g., CO₂ hospital house gas source). The second fluid inlet 346 may extend from a second end external to the second container 332 to a first end coupled to the second container 332. The alternative gas supply may be used to pressurize the second container 332 to supply lens wash to the endoscope 100 and/or to provide insufflation. A lumen extends through the second fluid inlet 346 for receiving a flow of gas therethrough. The lumen of the second fluid inlet 346 is in operative fluid communication with a top portion of the second container 332. The flow of the CO₂ through the system 300 may be similar to that described above. For example, in the neutral state, CO₂ gas flows through the second fluid inlet 346 into the second container 332, up the gas supply tubing 336 to the connector portion 265, up the gas feed line 240b in the umbilical 260, and is vented through the gas/water valve 140 to atmosphere. In the first position, the user closes off the vent hole in the gas/water valve 140, and the CO₂ gas is flowed through the second fluid inlet 346 into the second container 332, up the gas supply tubing 336 to the connector portion 265, through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the second reservoir 330 is maintained by delivering gas through the second fluid inlet 346. It is contemplated that the one-way valve 358 is in the closed configuration during delivery of the CO₂ gas to allow the container 332 to pressurize. In some instances, the one-way valve 358 may be configured to close without user intervention in response to the delivery of CO₂ to the second container 332. In some embodiments, the system 300 may include a branched connector (such as, but not limited to a “Y” or “T” connector) at the second fluid inlet 346 to allow either air or CO₂ to be used for pressurization or insufflation. It is further contemplated that the second fluid inlet 346 may include a pressure relief valve 362, such as, but not limited to, a 3-way stopcock, a clamp, or a spring-loaded valve, to vent pressure within the second container 332 and/or to block a flow of pressurized gas to the second container 332 during refilling of the second container 332, during procedure change-overs, and/or during equipment change-overs.

It is contemplated that the use of a flexible bag in place of a rigid bottle for the second reservoir 330 may reduce or eliminate the risk of air leaking from bottle and cap connections. This may eliminate the need for clinicians to attempt to remedy the leak by adjusting the cap and bottle assemblies or from discarding a cap and/or bottle if the leak cannot be remedied.

As the pressurized second container 332 is fluidly isolated from the first container 304 when the one-way valve 358 is closed, it is contemplated that the clinician may replace the first reservoir 302 with a new (full) reservoir without losing patient insufflation. Loss of patient insufflation may result in a loss of position of the endoscope 100 within the body. In current one or two bottle systems, it may not be possible to replace the water reservoirs without loss of patient insufflation.

If there is a need to replace the first reservoir 302 with a new full bag, for example when the first reservoir 302 is empty or near empty, the user may hang the new bag near the first reservoir 302 to be replaced. The user may then disengage the spike port adaptor 310 from the port 308b and insert the spike port adaptor 310 into a port of the new bag. This may be performed without requiring the clinician to bend or stoop to access the first reservoir 302. The port 308b may self-seal to prevent fluid leaks from the first reservoir 302 being replaced. This method of replacing the first reservoir 302 may have a lower risk of introducing contaminants into the systems relative to traditional bottle systems. For example, the change out method described herein may allow the first reservoir 302 to be changed out without having tubing dangling from a cap (as in a bottle system). Further, the system 300 may remain largely closed as the first reservoir 302 is changed out.

In some cases, it may be desirable to reduce the number of features that need to be managed during a procedure. For example, it may be desirable to provide lens wash without a separate system or a secondary fluid storage vessel. This may improve the user interface and/or reduce the amount of space needed for the system. In some cases, a pressure vessel system may be in selective fluid communication with a fluid source, such as, but not limited to, the first reservoir 302. The pressure vessel system may be filled with an amount of fluid sufficient to perform a lens wash operation prior to actuation of the gas/water valve 140. In some cases, the pressure vessel system may be filled in response to a user input. In other cases, the pressure vessel system may be filled without user input. In either case, the user need not monitor the fluid level in the pressure vessel system or replace the pressure vessel system

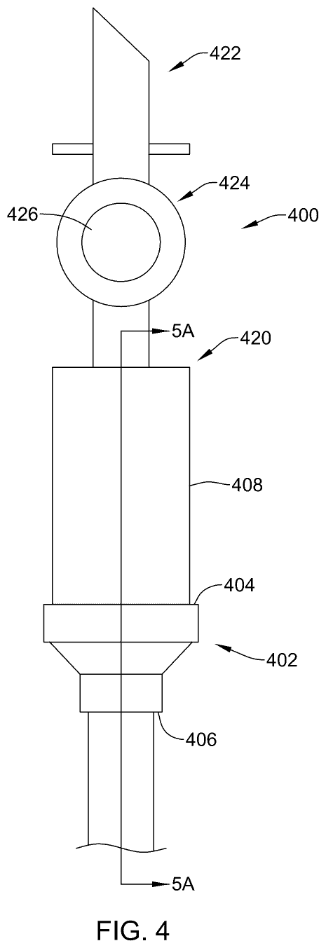

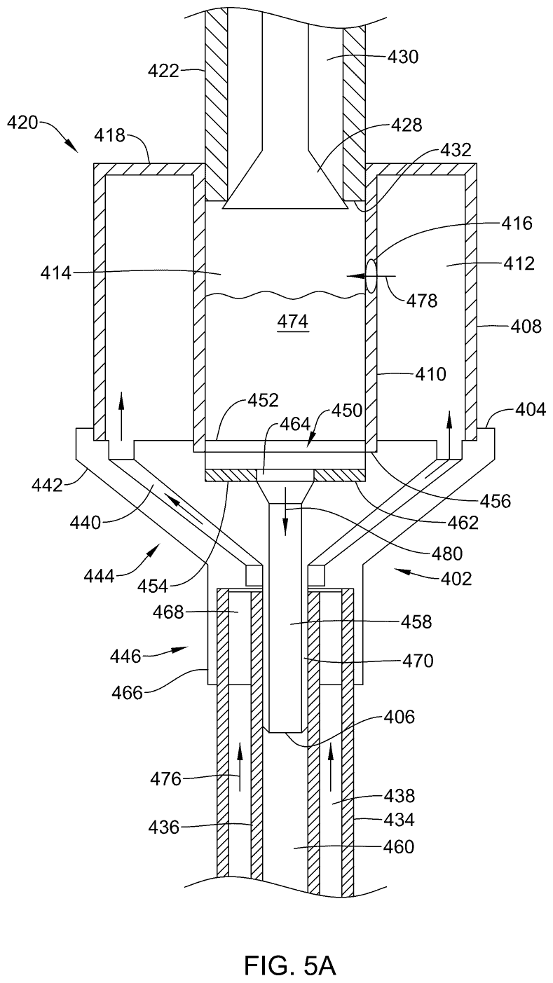

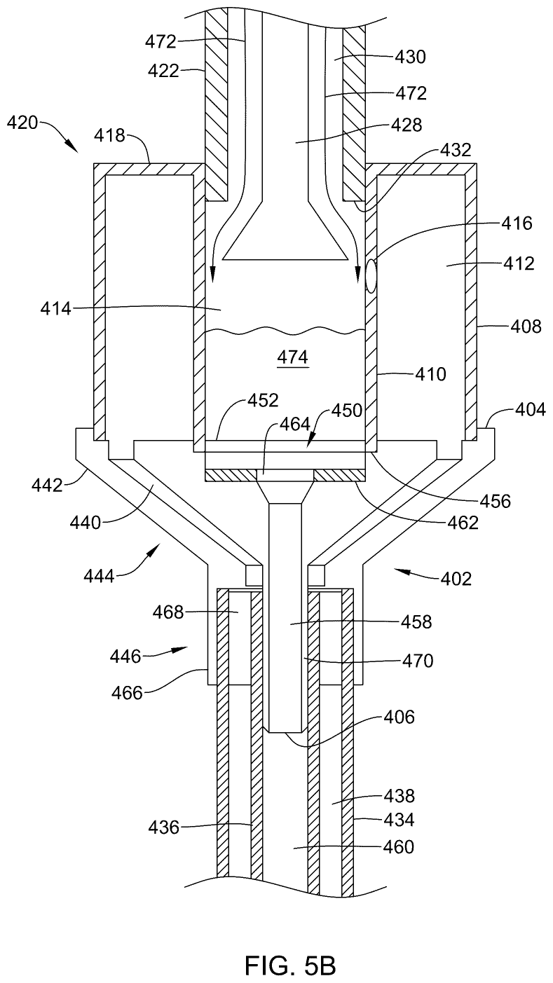

FIG. 4 is a side view of an illustrative pressure vessel system 400 that may be used in place of the secondary fluid reservoir 330 of FIG. 3. FIG. 5A is a partial cross-sectional view of the illustrative pressure vessel system 400 taken at line 5A-5A of FIG. 4, with the system 400 in a first, or closed configuration. FIG. 5B is a partial cross-sectional view of the illustrative pressure vessel system 400 in a second or refilling configuration. The pressure vessel system 400 may be configured to be fluidly and/or mechanically coupled to a fluid source, such as the first fluid reservoir 302 of FIG. 3.

Generally, the system 400 may include a tubing valve 402 configured to receive both air and water tubing at both a proximal end 404 and a distal end 406 of the tubing valve 402. The system 400 may include a proximal or upstream gas supply tubing 408 and a proximal or upstream lens wash supply (or water supply) tubing 410 coupled to the proximal end 404 of the tubing valve 402. The proximal gas and lens wash supply tubing 408, 410 may be coaxially arranged. For example, the proximal gas supply tubing 408 may define a lumen that is sufficiently large in diameter to encompass a smaller diameter proximal lens wash tubing 410, coaxially received within the proximal gas supply tubing 408, as well as provide air to the tubing valve 402 via an annular space 412 surrounding the proximal lens wash tubing 410 to pressurize a lumen 414 of the proximal lens wash supply tubing 410. In some embodiments, a wall of the proximal lens wash tubing 410 may include a one-way valve 416 configured to selectively fluidly couple the annular space 412 of the proximal gas supply tubing 408 with the lumen 414 of the proximal lens wash supply tubing 410. However, the one-way valve 416 is not required.

An annular cap or wall 418 may extend between the proximal gas supply tubing 408 and the proximal lens wash supply tubing 410 adjacent to a proximal or first end region 420 of the proximal gas and lens wash supply tubing 408, 410. The annular wall 418 may enclose the annular space 412 such that when air/gas is actively flowing, the air/gas is passed from the annular space 412, through the one-way valve 416, and into the lumen 414 of the proximal lens wash supply tubing 410. The proximal end region 420 of the proximal gas and lens wash supply tubing 408, 410 may be coupled to a spike port adaptor 422. The spike port adaptor 422 may be received within the port of a fluid source. For example, the spike port adaptor 422 may be inserted directly into one of the ports 308a, 308b of the first fluid reservoir 302 of the system 300 of FIG. 3. Alternatively, in the system 300 of FIG. 3, the second outlet leg 354 of the branched connector 350 may include a spike port, similar in form and function to the ports 308a, 308b described herein, configured to receive the spike port adaptor 422. The spike port adaptor 422 may define a lumen 430 configured to fluidly couple the first fluid reservoir 302, or other fluid source, with the lumen 414 of the lens wash supply tubing 410.

The spike port adaptor 422 may include a spring-loaded push button valve assembly 424 positioned in-line therewith. The push button valve assembly 424 may include a push button 426, or other actuation means, and a plunger 428. The plunger 428 may be biased to abut or contact a distal end 432 of the spike port adaptor 422, as shown in FIG. 5A, to provide a fluid-tight seal between the lumen 430 of the spike port adaptor 422 and the lumen 414 of the proximal lens wash supply tubing 410. For example, a biasing element, such as, but not limited to, a spring (not explicitly shown) may pull (or push) the plunger 428 into contact with the distal end 432 of the spike port adaptor 422. Actuation of the push button 426 may overcome the biasing force of the biasing element to move the plunger 428 distally away from the distal end 432 of the spike port adaptor 422, to a second or refilling configuration, as shown in FIG. 5B. This may allow fluid to flow from the first reservoir 302 through the lumen 430 of the spike port adaptor 422 and into the lumen 414 of the proximal lens wash supply tubing 410. Once the push button 426 is released, the biasing element may move the plunger 428 to the first or closed configuration (FIG. 5A) without further user intervention to prevent further flow of fluid from the first reservoir 302 into the lumen 414 of the proximal lens wash supply tubing 410.

The system 400 may further include a distal or downstream gas supply tubing 434 and a distal or downstream lens wash supply (or water supply) tubing 436 coupled to the distal end 406 of the tubing valve 402. The distal gas and lens wash supply tubing 434, 436 may be coaxially arranged. For example, the distal gas supply tubing 434 may define a lumen 438 that is sufficiently large in diameter to encompass a smaller diameter distal lens wash tubing 436, coaxially received within the proximal gas supply tubing 434, as well as provide air to proximal gas supply tubing 408. The distal gas and lens wash supply tubing 434, 436 may extend from a first end coupled to the tubing valve 402 to a second end (not explicitly shown) configured to be coupled to the gas/lens wash connection 290 on the outside of the connector portion 265.

The lumen 438 of the distal gas supply tubing 434 may be in fluid communication with one or more channels 440 extending through a body 442 of the tubing valve 402. The one or more channels 440 may extend from the proximal end 404 to the distal end of the tubing valve 402. The tubing valve 402 may including any number of channels 440 desired. For example, the tubing valve 402 may include one, two, three, four, or more channels 440. The channels 440 may be uniformly or eccentrically spaced about a circumference of the tubing valve 402, as desired. When the gas/water valve 140 is pressed downward to a second position, gas is blocked from exiting the valve, allowing pressure of the air passing from the air pump 215 (or alternative gas source, if so provided) to flow through the distal gas supply tubing 434, through the one or more channels 440 and into the annular space 412. Once the pressure in the annular space 412 exceeds a predetermined threshold, air/gas may pass through the one-way valve 416 and into the lumen 414 of the proximal lens wash supply tubing 410.

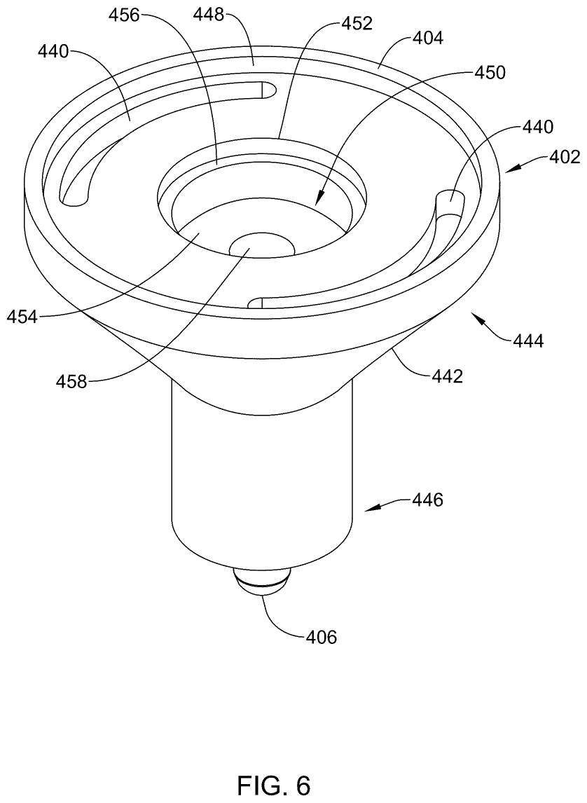

Referring additionally to FIG. 6, which illustrates a proximal perspective view of the tubing valve 402, the valve body 442 may include a proximal end region 444 and a distal end region 446. In some examples, the proximal end region 444 may have a greater diameter than the distal end region 446, although this is not required. In some examples, the proximal end region 444 may have a truncated conical shape with the diameter reducing in the distal direction. It is contemplated that the proximal end region 444 may take other shapes, as desired. In other examples, the proximal end region 444 and the distal end region 446 may have similar diameters. In yet other examples, the proximal end region 444 may have a smaller diameter than the distal end region 446

The proximal end region 444 may be sized and shaped to couple to the proximal gas supply tubing 408 and the proximal lens wash supply tubing 410 while the distal end region 446 may be sized and shaped to be coupled to the distal gas supply tubing 434 and the distal lens wash supply tubing 436. In some embodiments, the proximal end 404 of the tubing valve 402 may include a raised annular lip 448 configured to surround an outer surface of the proximal gas supply tubing 408. However this is not required. In other embodiments, the proximal gas supply tubing 408 may be received within an annular recess or groove. The proximal end region 444 of the body 442 may further include a central recess 450. The recess 450 may have a generally circular cross-sectional shape. However, this is not required. The recess 450 may take other shapes, as desired. The diameter (or cross-sectional dimension) may vary along a length of the recess 450. For example, the recess 450 may have a first diameter adjacent to a proximal end 452 of the recess 450 and a second diameter adjacent to a distal end 454 of the recess 450. The first diameter may be greater than the second diameter. In some examples, the diameter may change in an abrupt manner to define a radially inwardly extending ledge or rim 456. The proximal lens wash supply tubing 410 may be received within the recess 450 and in some examples, may be positioned against or adjacent to the rim 456. The channels 440 may be positioned between the recess 450 and the annular lip 448 to fluidly couple to the annular space 412 of the proximal gas supply tubing 408.

A central lumen 458 may extend from the distal end 454 of the recess 450 to the distal end 406 of the body 442 of the tubing valve 402. The central lumen 458 may be in selective fluid communication with the lumen 414 of the proximal lens wash supply tubing 410 and a lumen 460 of the distal lens wash supply tubing 436. A cross-slit or snowflake slit valve 462 (see, FIGS. 5A and 5B) may be positioned within the recess 450 adjacent to the distal end 454 thereof. The valve 462 may be formed from a flexible material such as, but not limited to, silicone, thermoplastic elastomers, or the like. The valve 462 may include one or more slits 464 extending through a thickness thereof. When the pressure inside of the lumen 414 of the proximal lens wash supply tubing 410 exceeds a predetermined threshold, water or fluid may pass from the lumen 414 of the proximal lens wash supply tubing 410 through the one or more slits 464 of the valve 462 (e.g., the walls of the one or more slits 464 may move away from one another) and into the lumen 460 of the distal lens wash supply tube 436 to be supplied to the endoscope. When the pressure inside of the lumen 414 of the proximal lens wash supply tubing 410 is below a predetermined threshold, the sides of the one or more slits 464 may move towards one another and the valve 462 may return to a closed configuration in which water or fluid is prevented from exiting the lumen 414 of the proximal lens wash supply tubing 410.

Returning to FIGS. 5A and 5B, the distal end region 446 of the tubing valve 402 may include an outer tubular member 466 defining a lumen or cavity 468 and an inner tubular member 470 disposed radially inwards of the outer tubular member 466. The inner tubular member 470 may define a least a portion of the central lumen 458. The distal gas supply tubing 434 may be received within the cavity 468 of the outer tubular member 466. The one or more channels 440 may be in fluid communication with the cavity 468 of the outer tubular member 466. The distal gas supply tubing 434 may be positioned within the cavity 468 to fluidly couple the lumen 438 of the distal gas supply tubing 434 with the one or more channels 440. The distal lens wash supply tubing 436 may be disposed over and/or coupled to the inner tubular member 470 to fluidly couple the lumen 460 of the distal lens wash supply tubing 436 with the central lumen 458.

When lens wash is desired, the clinician may actuate the push button 426. This may move the plunger 428 away from the distal end 432 of the spike port adaptor 422. Water or other fluid may flow from the first reservoir 302 through the lumen 430 of the spike port adaptor 422 and into the lumen 414 of the proximal lens wash supply tubing 410, as shown at arrows 472 of FIG. 5B. When the desired amount of water 474 is within the lumen 414 of the proximal lens wash supply tubing 410, the clinician may release the push button 426. The biasing mechanism may return the plunger 428 to the closed configuration (FIG. 5A) to fluidly isolate the lumen 414 of the proximal lens wash supply tubing 410 from the lumen 430 of the spike port adaptor 422. When the gas/water valve 140 is pressed downward to a second position, gas is blocked from exiting the valve, allowing pressure of the air passing from the air pump 215 (or alternative gas source, if so provided) to flow through the distal gas supply tubing 434, through the one or more channels 440 and into the annular space 412, as shown at arrows 476 in FIG. 5A. Once the pressure in the annular space 412 exceeds a predetermined threshold, air/gas may pass through the one-way valve 416 and into the lumen 414 of the proximal lens wash supply tubing 410, as shown at arrow 478 of FIG. 5A. When the pressure inside of the lumen 414 of the proximal lens wash supply tubing 410 exceeds a predetermined threshold, water or fluid 474 may pass from the lumen 414 of the proximal lens wash supply tubing 410 through the one or more slits 464 of the valve 462 (e.g., the walls of the one or more slits 464 may move away from one another) and into the lumen 460 of the distal lens wash supply tube 436 to be supplied to the endoscope, as shown at arrow 480 of FIG. 5A. Releasing the gas/water valve 140 may allow the gas/air to vent from the proximal and/or distal gas supply tubing 408, 464 and the system 400 to return to atmospheric pressure. When the pressure inside of the lumen 414 of the proximal lens wash supply tubing 410 is below a predetermined threshold, the sides of the one or more slits 464 may move towards one another and the valve 462 may return to a closed configuration in which water or fluid is prevented from exiting the lumen 414 of the proximal lens wash supply tubing 410. The one-way valve 416 may prevent water 474 from moving from the lumen 414 of the proximal lens wash supply tubing 410 to the proximal gas supply tubing 408.

It is contemplated that the proximal gas supply tubing 408 may have an outer diameter and/or wall thickness greater than the distal gas supply tubing 434. Similarly, the proximal lens wash supply tubing 410 may have an outer diameter and/or wall thickness greater than the distal lens wash supply tubing 436. In some examples, the proximal gas supply tubing 408 and/or the proximal lens wash supply tubing 410 may be formed from a rigid material configured to withstand the pressure required to supply the lens wash fluid. In other examples, the proximal lens wash supply tubing 410 may be formed from a flexible or deformable material, such as, but not limited to, silicone, thermoplastic elastomers, or the like. When the proximal lens wash supply tubing 410 is formed from a flexible material, the one-way valve may be omitted. As the pressure increases within the annular space 412, the walls of the flexible lens wash supply tubing 410 may move radially inwards thus increasing the pressure of the lumen 414 of the proximal lens wash supply tubing 410 and forcing the water 474 through the valve 462 and into the distal lens wash supply tubing 436.

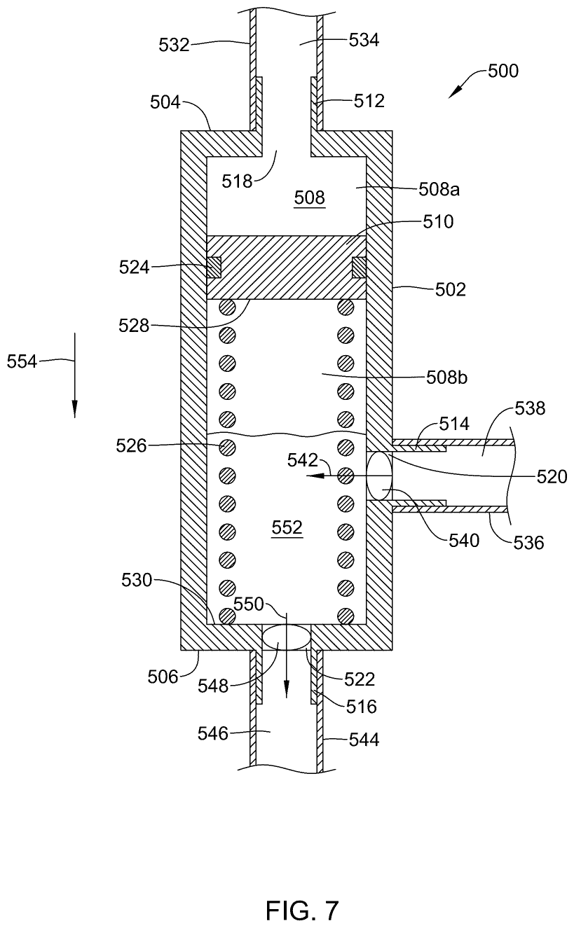

FIG. 7 is a cross-sectional view of another illustrative pressure vessel system 500 that may be used in place of the secondary fluid reservoir 330 of FIG. 3. The system 500 may include a manifold housing 502 extending from a first end 504 to a second end 506. As used herein, a “manifold” is a structure having two or more openings for making fluid connections. In some embodiments, the housing 502 may have a generally cylindrical shape. However, the housing 502 may take other shapes, as desired. The housing 502 may define a cavity 508 therein. A piston 510 may divide the cavity 508 into a first chamber 508a and a second chamber 508b. The housing 502 may include a first port 512, a second port 514, and a third port 516. The ports 512, 514, 516 may be formed as a monolithic structure with the housing 502. However, this is not required. In some embodiments, the ports 512, 514, 516 may be separately formed and subsequently coupled with a respective opening 518, 520, 522 extending through a sidewall of the housing 502. The ports 512, 514, 516 may be generally tubular structures with each port 512, 514, 516 defining a lumen extending therethrough to define a fluid inlet or outlet. The lumens of the ports 512, 514, 516 may be configured to selectively fluidly couple the cavity 508 of the housing 502 with another component, such as, but not limited to, a fluid or water supply tube and/or a gas supply tube. In the illustrated embodiment, the first port 512 is positioned adjacent to the first end 504, the second port 514 is positioned within a lateral sidewall, and the third port 516 is positioned adjacent to the second end 506. However, this is not required. The ports 512, 514, 516 may be positioned in other locations, as desired.

The piston 510 may include a gasket, O-ring, or other sealing member 524 extending about a perimeter thereof. The sealing member 524 may fluidly isolate the first chamber 508a from the second chamber 508b. A biasing member 526, such as, but not limited to, a spring, may be positioned within the second chamber 508b. The biasing member 526 may extend between a second end 528 of the piston 510 and an inner surface 530 of the housing 502 adjacent to the second end 506 thereof. The biasing member 526 may be configured to bias the piston 510 towards the first end 504 of the housing 502 in the absence of an external biasing force.

The system 500 may further include a gas supply tubing 532 coupled to the first port 512. The distal gas supply tubing 532 may extend from a first end coupled to the first port 512 to a second end (not explicitly shown) configured to be coupled to the gas/lens wash connection 290 on the outside of the connector portion 265. The gas supply tubing 532 may define a lumen 534 configured to supply air or an alternative gas (e.g., from the air pump 215 or an alternative gas source) to the first chamber 508a of the housing 502.

A fluid supply tubing 536 may be coupled to the second port 514. The fluid supply tubing 536 may extend from a first end coupled to the second port 514 to a second end (not explicitly shown) configured to be coupled to a fluid supply source. In some embodiments, the fluid supply tubing 536 may be configured to be coupled to one of the ports 308a, 308b of the first fluid reservoir 302 of the system 300 of FIG. 3. For example, the fluid supply tubing 536 may include a spike port adaptor (not explicitly shown) configured to be received within one of the ports 308a, 308b. Alternatively, in the system 300 of FIG. 3, the second outlet leg 354 of the branched connector 350 may include a spike port, similar in form and function to the ports 308a, 308b described herein, configured to receive a spike port adaptor of the fluid supply tubing 536. The fluid supply tubing 536 may define a lumen 538 configured to supply water or fluid to the second chamber 508b of the housing 502. A one-way valve 540, or other flow control mechanism, may be positioned in-line with the lumen 538 of the fluid supply tubing 536. In some examples, the one-way valve 540 may be positioned within the second opening 520 of the housing 502. In other examples, the one-way valve 540 may be positioned within the second port 514 or within the fluid supply tubing 536. The one-way valve 540 may be configured to allow a flow of fluid into the second chamber 508b, as shown at arrow 542, while preventing fluid from exiting the second chamber 508b via the second port 514.

A lens wash supply tubing 544 may be coupled to the third port 516. The lens wash supply tubing 544 may extend from a first end coupled to the third port 516 to a second end (not explicitly shown) configured to be coupled to the gas/lens wash connection 290 on the outside of the connector portion 265 to supply lens wash fluid to the endoscope 100. The lens wash supply tubing 544 may define a lumen 546 configured to supply water or fluid from the second chamber 508b of the housing 502 to the endoscope 100. A one-way valve or other flow control mechanism 548 may be positioned in-line with the lumen 546 of the lens wash supply tubing 544. In some examples, the one-way valve 548 may be positioned within the third opening 522 of the housing 502. In other examples, the one-way valve 548 may be positioned within the third port 516 or within the lens wash supply tubing 544. The one-way valve 548 may be configured to allow a flow of fluid from the second chamber 508b, as shown at arrow 550, while preventing fluid from entering the second chamber 508b via the third port 516.

When lens wash is desired, the clinician may press the gas/water valve 140 downward to a second position. Gas is blocked from exiting the valve, allowing pressure of the air passing from the air pump 215 (or alternative gas source, if so provided) to flow through the gas supply tubing 532 and into the first chamber 508a. As the air is supplied to the first chamber 508a, the pressure inside the first chamber 508a increases. Once the pressure inside the first chamber 508a is sufficient to overcome the biasing force of the biasing member 526, the biasing member 526 is compressed and the piston 510 is moved towards the second end 506 of the housing 502, as shown at arrow 554. The movement of the piston 510 may displace the water or fluid 552 within the second chamber 508b causing the water 552 to exit the second chamber 508b via the third port 516 to be supplied as lens wash fluid. When the gas/water valve 140 is released, air/gas may exit the first chamber 508a (e.g., via the first port 512). As the air/gas exits the first chamber 508a, the biasing member 526 moves the piston 510 towards the first end 504 of the housing 502. As the piston 510 is moved towards the first end 504 of the housing 502, water or other fluid (e.g., from the first fluid reservoir 302) is sucked or pulled into the second chamber 508b via the second port 514. The sealing member 524 may prevent air/gas from entering the second chamber 508b and may prevent water 552 from entering the first chamber 508a.

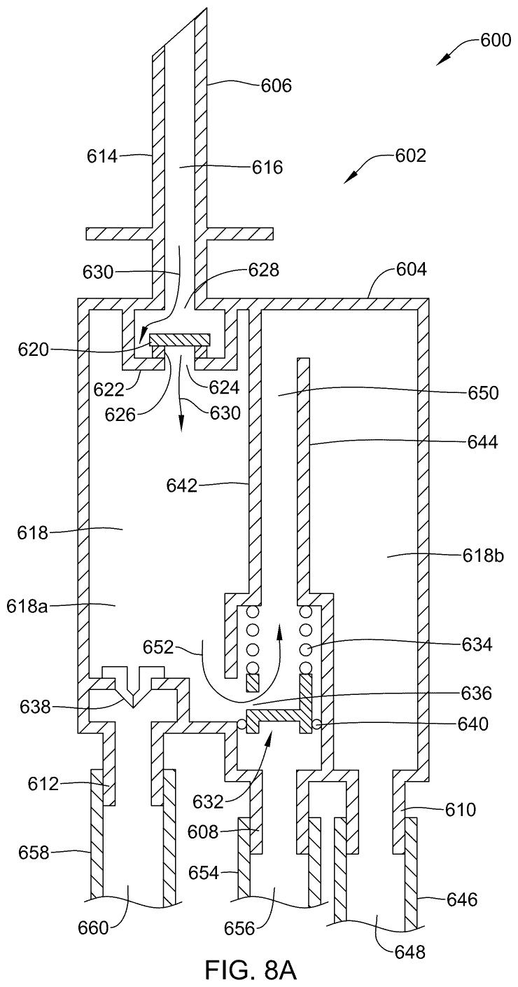

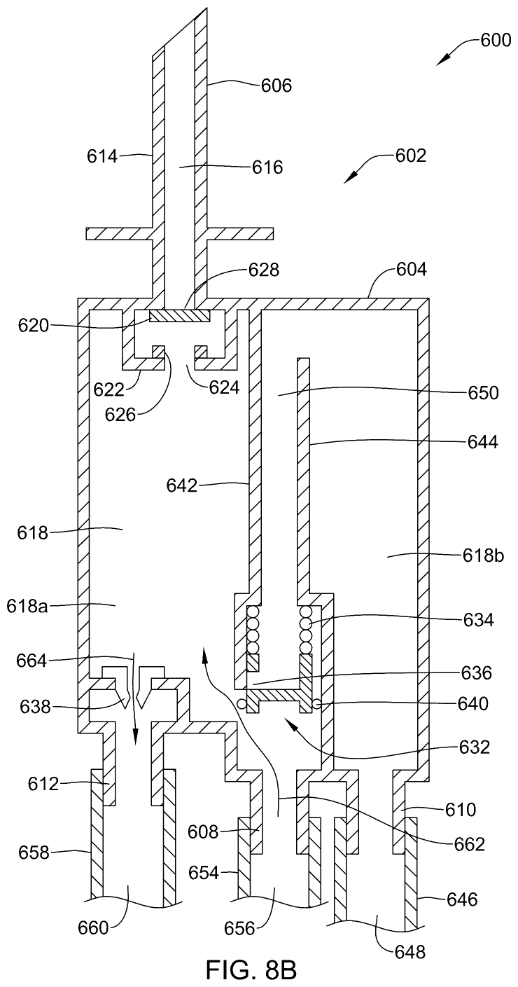

FIG. 8A is a cross-sectional view of another illustrative pressure vessel system 600 in a first configuration that may be used in place of the secondary fluid reservoir 330 of FIG. 3. FIG. 8B is a cross-sectional view of the illustrative pressure vessel system 600 of FIG. 8A in a second configuration. Generally, the pressure vessel system 600 may be configured to be fluidly coupled to a fluid source, such as, but not limited to the first fluid reservoir 302 of FIG. 3. The pressure vessel system 600 may be configured to supply fluid to the endoscope for both irrigation and lens wash.

The pressure vessel system 600 may include a manifold assembly 602 including a manifold housing 604. As used herein, a “manifold” is a structure having two or more openings for making fluid connections. The manifold housing 604 may include a first fluid inlet 606, a second fluid inlet 608, a first fluid outlet 610, and a second fluid outlet 612. In some embodiments, the inlets 606, 608 and/or outlets 610, 612 may be tubular ports or openings in the manifold housing 604 configured to selectively fluidly couple the manifold housing 604 with another component, such as, but not limited to, a fluid or water supply tube and/or a gas supply tube. The first fluid inlet 606 may include a spike port adaptor 614. The spike port adaptor 614 may be received within the port of a fluid source. For example, the spike port adaptor 614 may be inserted directly into one of the ports 308a, 308b of the first fluid reservoir 302 of the system 300 of FIG. 3. The spike port adaptor 614 may define a lumen 616 configured selectively to fluidly couple the first fluid reservoir 302, or other fluid source, with a cavity 618 of the manifold housing 604.

A flow control member 620 may be positioned within the cavity 618 of the manifold housing 604 adjacent to the first fluid inlet 606. The flow control member 620 may be configured to selectively block or occlude a first end opening 628 of the first fluid inlet 606. When the flow control member 620 is disposed against or over the first end opening 628 of the first fluid inlet 606, fluid is prevented from flowing from the fluid source (e.g., first reservoir 302) into the cavity 618 of the manifold housing 604 (see, for example, FIG. 8B). In some cases, the flow control member 620 may be a floating stopper held against the first end opening 628 of the first fluid inlet 606. The flow control member 620 may be disposed against or over the first end opening 628 of the first fluid inlet 606 by an increase in the pressure within the manifold housing 604. When the pressure is vented (e.g., by releasing the gas/water valve 140), the pressure in the manifold housing 604 may decrease allowing the flow control member 620 to be displaced away from first end opening 628 of the first fluid inlet 606 such that fluid can flow into the cavity 618 of the manifold housing 604, as shown at arrow 630 in FIG. 8A. A stop mechanism 622 may be positioned adjacent to the flow control member 620 to prevent the flow control member 620 from dropping to a bottom of the manifold housing 604. The stop mechanism 622 may include an opening 624 that is smaller than the flow control member 620 to allow fluid to pass while maintaining the flow control member 620 adjacent to first end opening 628 of the first fluid inlet 606. The flow control member 620 may be spaced from the opening 624 by one or more circumferentially spaced ribs 626 extending from the stop mechanism 622. The one or more circumferentially spaced ribs 626 may have gaps or openings therebetween to allow fluid to pass between the flow control member 620 and the stop mechanism 622. In other examples, the flow control member 620 may be a one-way valve that allows fluid to flow into the cavity 618 of the manifold housing 604 when the pressure of the cavity 618 of the manifold housing 604 falls below a predetermined threshold.

The cavity 618 of manifold housing 604 may be separated into a first chamber 618a and a second chamber 618b by a first internal wall 642, a second internal wall 644, and a spring-loaded linear displacement valve 632 movable between a first configuration (FIG. 8A) and a second configuration (FIG. 8B). A channel 650 may fluidly couple the first chamber 618a and the second chamber 618b with the linear displacement valve 632 selectively coupling the first chamber 618a and the second chamber 618b. The linear displacement valve 632 may be biased into a first configuration by a spring 634, or other biasing member. When the linear displacement valve 632 is in the first configuration, an opening 636 of the linear displacement valve 632 may fluidly couple the first chamber 618a and the second chamber 618b. When the linear displacement valve 632 is in the second configuration, the linear displacement valve 632 may fluidly isolate the second chamber 618b from the first chamber 618a. A gasket, O-ring, or other sealing member 640 may be disposed between an outer surface of the linear displacement valve 632 and the manifold housing 602 to fluidly isolate the second fluid inlet 608 from the cavity 618 of the manifold housing 604 when the linear displacement valve 632 is in the first configuration.

A one-way valve 638 may be positioned between the first chamber 618a and the second fluid outlet 612. The one-way valve 638 may be configured allow fluid to exit the first chamber 618a while preventing water from re-entering the first chamber 618a via the second fluid outlet 612. The one-way valve 638 may be configured to open at a predetermined pressure greater than atmospheric pressure such that fluid may be selectively delivered to the first fluid outlet 610 or the second fluid outlet 612, as will be described in more detail herein.

The first fluid outlet 610 may be configured to be coupled to an upstream irrigation supply tubing 646 defining a lumen 648 for supplying water or other fluid to an endoscope. The upstream irrigation supply tube 646 extends from a second end region (not explicitly shown) external to the manifold housing 604 and positioned within a pump head of the peristaltic irrigation pump to a first end coupled to the first outlet port 610 to fluidly couple the interior of the manifold housing 604 with the lumen 648 of the upstream irrigation supply tube 646. The second end of the upstream irrigation supply tubing 646 is configured to be fluidly coupled with an irrigation lumen of the endoscope 100. When irrigation water is required, fluid is pumped from the cavity 618 of the manifold housing 604 by operating the irrigation pump 315, such as by depressing a footswitch (not shown), and flows from the first reservoir 302 (arrow 630), through the first chamber 618a of the manifold housing 604, through the opening 638 in the linear displacement valve 632 (arrow 652), through a channel 650 defined by the first and second interior walls 642, 644, through the second chamber 618b, and into the upstream irrigation supply tubing 646. With the flow control member 620 in the open configuration, fluid may continually flow from the fluid source (e.g., first reservoir 302) as fluid is removed from the cavity 618 of the manifold housing 604 for irrigation.

The second fluid inlet 608 may be configured to be coupled to a gas supply tubing 654. The gas supply tubing 654 may extend from a first end coupled to the second fluid inlet 608 to a second end (not explicitly shown) configured to be coupled to the gas/lens wash connection 290 on the outside of the connector portion 265. The gas supply tubing 654 may define a lumen 656 configured to supply air or an alternative gas (e.g., from the air pump 215 or an alternative gas source) to the first chamber 618a of the manifold housing 604.

A lens wash supply tubing 658 may be coupled to the second fluid outlet 612. The lens wash supply tubing 658 may extend from a first end coupled to the second fluid outlet 612 to a second end (not explicitly shown) configured to be coupled to the gas/lens wash connection 290 on the outside of the connector portion 265 to supply lens wash fluid to the endoscope 100. The lens wash supply tubing 658 may define a lumen 660 configured to supply water or fluid from the first chamber 618a of the manifold housing 604 to the endoscope 100.

Referring additionally to FIG. 8B, when lens wash is desired, the clinician may press the gas/water valve 140 downward to a second position. Gas is blocked from exiting the valve, allowing pressure of the air passing from the air pump 215 (or alternative gas source, if so provided) to flow through the gas supply tubing 654 and into the second fluid inlet 608. The flow of gas may push against the linear displacement valve 632. The pressure of the gas may move the linear displacement valve 632 towards the spring 634 causing the spring 634 to compress. A portion of the linear displacement valve 632 may block fluid flow through the channel 650 thus causing the air/gas to enter the first chamber 618a, as shown at arrow 662. The linear displacement valve 632 may fluidly isolate the channel 650 and the second chamber 618b from the air/gas. As the air/gas enters the first chamber 618a, the pressure of the first chamber 618a may increase. This may cause the flow control member 620 to move towards first end opening 628 of the first fluid inlet 606 to block the first end opening 628. This may allow the pressure to further increase and the one-way valve 638 disposed between the first chamber 618b and the second fluid outlet 612 may open thus allowing fluid to exit the second fluid inlet 612 and flow into the lumen 660 of the lens wash supply tubing 658, as shown at arrow 664. When the gas/water valve 140 is released, air/gas may exit the first chamber 618a allowing the one-way valve 638 to close and the flow control member 620 to move away from the first end opening 628 of the first fluid inlet 606 to allow fluid to enter the cavity 618 of the manifold housing 604. It is contemplated that pulling irrigation fluid through first and second chambers 618a, 618b when the user calls for irrigation may refill the first chamber 618a while removing most of the air or CO₂ that remains in the first chamber 618a after a lens wash cycle.

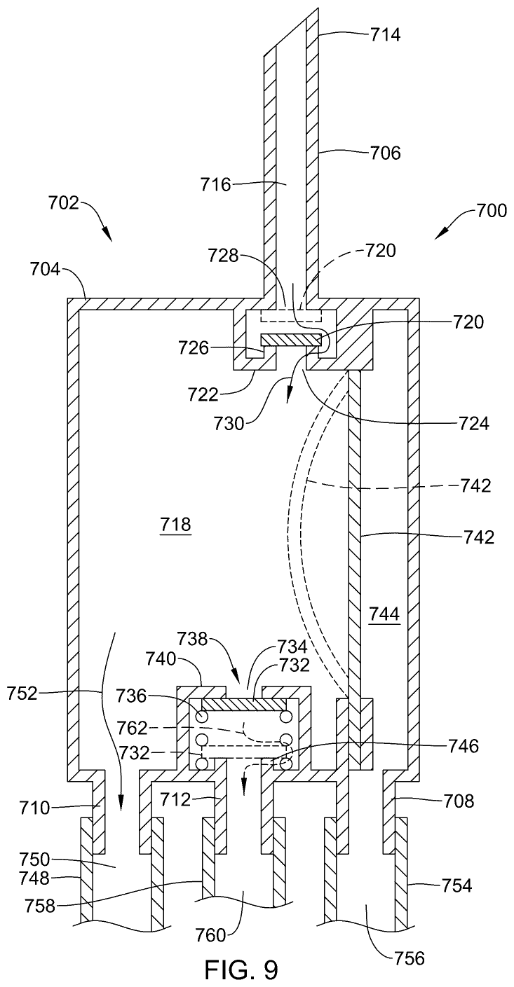

FIG. 9 is a cross-sectional view of another illustrative pressure vessel system 700 in a first configuration that may be used in place of the secondary fluid reservoir 330 of FIG. 3. Generally, the pressure vessel system 700 may be configured to be fluidly coupled to a fluid source, such as, but not limited to the first fluid reservoir 302 of FIG. 3. The pressure vessel system 700 may be configured to supply fluid to the endoscope for both irrigation and lens wash.

The pressure vessel system 700 may include a manifold assembly 702 including a manifold housing 704. As used herein, a “manifold” is a structure having two or more openings for making fluid connections. The manifold housing 704 may include a first fluid inlet 706, a second fluid inlet 708, a first fluid outlet 710, and a second fluid outlet 712. In some embodiments, the inlets 706, 708 and/or outlets 710, 712 may be tubular ports or openings in the manifold housing 704 configured to selectively fluidly couple the manifold housing 704 with another component, such as, but not limited to, a fluid or water supply tube and/or a gas supply tube. The first fluid inlet 706 may include a spike port adaptor 714. The spike port adaptor 714 may be received within the port of a fluid source. For example, the spike port adaptor 714 may be inserted directly into one of the ports 308a, 308b of the first fluid reservoir 302 of the system 300 of FIG. 3. The spike port adaptor 714 may define a lumen 716 configured to selectively fluidly couple the first fluid reservoir 302, or other fluid source, with a first chamber 718 of the manifold housing 704.

A flow control member 720 may be positioned within the first chamber 718 of the manifold housing 704 adjacent to the first fluid inlet 706. The flow control member 720 may be configured to selectively block or occlude a first end opening 728 of the first fluid inlet 706. When the flow control member 720 is disposed against or over the first end opening 728 of the first fluid inlet 706, fluid is prevented from flowing from the fluid source (e.g., first reservoir 302) into the first chamber 718 of the manifold housing 704. In some cases, the flow control member 720 may be a floating stopper held against the first end opening 728 of the first fluid inlet 706. The flow control member 720 may be disposed against or over the first end opening 728 of the first fluid inlet 706 by the pressure within the manifold housing 704. When the pressure is vented (e.g., by releasing the gas/water valve 140), the pressure in the first fluid inlet 706 may cause the flow control member 720 to be displaced away from first end opening 728 of the first fluid inlet 706 such that fluid can flow into the first chamber 718 of the manifold housing 704, as shown at arrow 730. A stop mechanism 722 may be positioned adjacent to the flow control member 720 to prevent the flow control member 720 from dropping to a bottom of the manifold housing 704. The stop mechanism 722 may include an opening 724 that is smaller than the flow control member 720 to allow fluid to pass while maintaining the flow control member 720 adjacent to first end opening 728 of the first fluid inlet 706. The flow control member 720 may be spaced from the opening 724 by one or more circumferentially spaced ribs 726 extending from the stop mechanism 722. The one or more circumferentially spaced ribs 726 may have gaps or openings therebetween to allow fluid to pass between the flow control member 720 and the stop mechanism 722. In other examples, the flow control member 720 may be a one-way valve that allows fluid to flow into the first chamber 718 of the manifold housing 704 when the pressure of the first chamber 718 of the manifold housing 704 falls below a predetermined threshold.

A diaphragm 742 may fluidly isolate the first chamber 718 of manifold housing 704 from a second chamber 744 of the manifold housing 704. The diaphragm 742 may be formed from a flexible or deformable material that can be stretched and subsequently return to an original configuration. For example, in response to an increase in air volume in the second chamber 744, the diaphragm 742 may expand into the first chamber 718 reducing the volume of the first chamber 718. The diaphragm 742 may take many forms. In one illustrative example, the diaphragm 742 may be cylindrical and positioned along an inside wall of a cylindrical manifold housing 704, thus allowing pressurization from all sides and facilitating a larger displacement efficiency.

A one-way valve 738 may be positioned between the first chamber 718 and the second fluid outlet 712. The one-way valve 738 may be configured allow fluid to exit the first chamber 718 while preventing water from re-entering the first chamber 718 via the second fluid outlet 712. The one-way valve 738 may be configured to open at a predetermined pressure greater than atmospheric pressure such that fluid may be selectively delivered to the first fluid outlet 710 or the second fluid outlet 712, as will be described in more detail herein.

In some examples, the one-way valve 738 may include a flow control member 732 positioned adjacent to the second fluid outlet 712. The flow control member 732 may be configured to selectively block or occlude an opening 734 between the first chamber 718 and the second fluid outlet 712. When the flow control member 732 is disposed against or over the opening 732, fluid is prevented from flowing from the first chamber 718 and through the second fluid outlet 712. In some cases, the flow control member 732 may be a floating stopper held against the opening 734 with a biasing member, such as, but not limited to a spring 736. The flow control member 732 may be biased away from the opening 734 by an increase in the pressure within the first chamber 718 of the manifold housing 704. When the pressure is vented (e.g., by releasing the gas/water valve 140), the pressure in the first chamber 718 may decrease causing the flow control member 732 to be displaced towards the opening 734 to prevent fluid from exiting via the second fluid outlet 712. A first stop mechanism 740 may be positioned adjacent to the flow control member 732 to limit movement of the flow control member 732 into the first chamber 718. A second stop mechanism 746 may be spaced from the first stop mechanism 740. The second stop mechanism 746 may include one or more circumferentially spaced ribs. The one or more circumferentially spaced ribs may have gaps or openings therebetween to allow fluid to pass between the flow control member 732 and the second stop mechanism 746. In other examples, the flow control member 732 may be a one-way valve that allows fluid to flow from the first chamber 718 of the manifold housing 704 when the pressure of the first chamber 718 of the manifold housing 704 exceeds a predetermined threshold.

The first fluid outlet 710 may be configured to be coupled to an upstream irrigation supply tubing 748 defining a lumen 750 for supplying water or other fluid to an endoscope. The upstream irrigation supply tubing 748 extends from a second end region (not explicitly shown) external to the manifold housing 704 and positioned within a pump head of the peristaltic irrigation pump to a first end coupled to the first outlet port 710 to fluidly couple the interior of the manifold housing 704 with the lumen 750 of the upstream irrigation supply tube 748. The second end of the upstream irrigation supply tube 748 is configured to be fluidly coupled with an irrigation lumen of the endoscope 100. When irrigation water is required, fluid is pumped from the first chamber 718 of the manifold housing 704 by operating the irrigation pump, such as by depressing a footswitch (not shown), and flows from the first reservoir 302 (arrow 730), through the first chamber 718 of the manifold housing 704 (arrow 752), through the first fluid outlet 710, and into the upstream irrigation supply tubing 748. With the first flow control member 720 in the open configuration, fluid may continually flow from the fluid source (e.g., first reservoir 302) as fluid is removed from the first chamber 718 of the manifold housing 704 for irrigation. The pressure of the fluid flowing through the first chamber 718 to provide irrigation fluid may be less than the pressure required to move (or open) the second flow control member 732. Thus, as irrigation fluid is pumped into the endoscope fluid the second flow control member 732 prevents fluid from exiting the second fluid outlet 712.

The second fluid inlet 708 may be configured to be coupled to a gas supply tubing 754. The gas supply tubing 754 may extend from a first end coupled to the second fluid inlet 708 to a second end (not explicitly shown) configured to be coupled to the gas/lens wash connection 290 on the outside of the connector portion 265. The gas supply tubing 754 may define a lumen 756 configured to supply air or an alternative gas (e.g., from the air pump 215 or an alternative gas source) to the second chamber 744 of the manifold housing 704.

A lens wash supply tubing 758 may be coupled to the second fluid outlet 712. The lens wash supply tubing 758 may extend from a first end coupled to the second fluid outlet 712 to a second end (not explicitly shown) configured to be coupled to the gas/lens wash connection 290 on the outside of the connector portion 265 to supply lens wash fluid to the endoscope 100. The lens wash supply tubing 758 may define a lumen 760 configured to supply water or fluid from the first chamber of the manifold housing 704 to the endoscope 100.

When lens wash is desired, the clinician may press the gas/water valve 140 downward to a second position. Gas is blocked from exiting the valve, allowing pressure of the air passing from the air pump 215 (or alternative gas source, if so provided) to flow through the gas supply tubing 754 and into the second chamber 744 via the second fluid inlet 708. The pressure of the gas may increase the volume of the second chamber 744 and move the diaphragm 742 towards the first chamber 718. The pressure of the first chamber 718 may be increased as the volume thereof is decreased. The increase in the pressure of the first chamber 718 may move the first flow control member 720 to block the first end opening 728 of the first fluid inlet 706 preventing additional water from entering the first chamber 718. Further, the increase in the pressure of the first chamber 718 may move the second flow control member 732 away from the opening 734 to allow fluid to flow from the first chamber 718, through the second fluid outlet 712 and into the lens wash supply tubing 758, as shown at arrow 762. When the gas/water valve 140 is released, air/gas may exit the second chamber 744 and the diaphragm 742 may return to its original configuration creating a negative pressure in the first chamber 718. This may cause the first flow control member 720 to return to an open configuration once again allowing fluid to enter the first chamber 718 from the lumen 716 of the first fluid inlet 706 and the second flow control member 7832 to return to the closed configuration once again preventing fluid from exiting the first chamber 718 via the second fluid outlet 712.

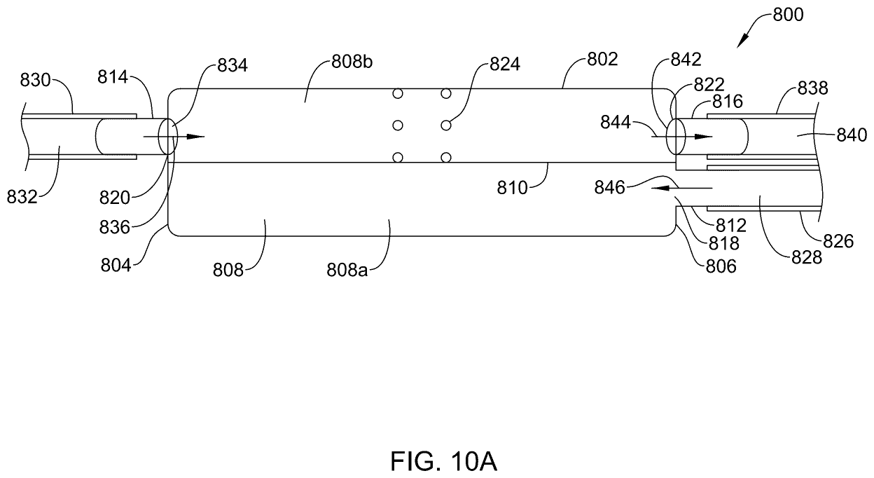

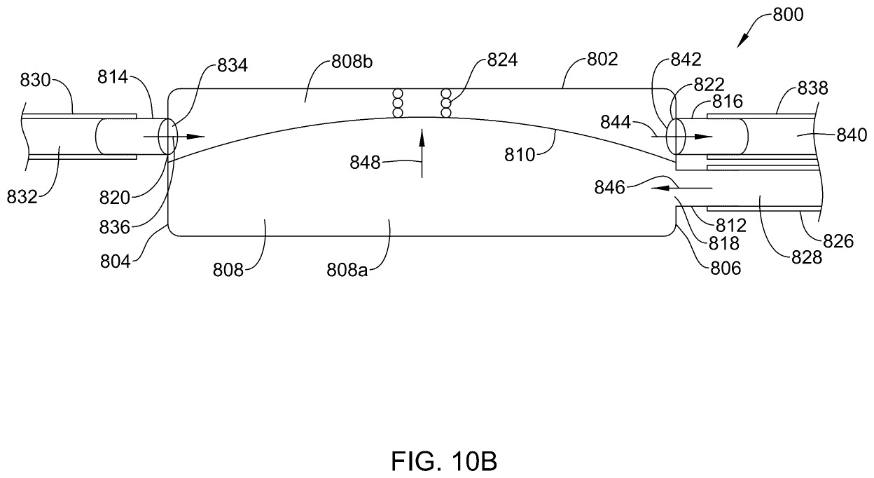

FIG. 10A is a schematic cross-sectional view of another illustrative pressure vessel system 800 in a first configuration that may be used in place of the secondary fluid reservoir 330 of FIG. 3. FIG. 10B is a schematic cross-sectional view of the illustrative system 800 of FIG. 10A in a second configuration. The system 800 may include a manifold housing 802 extending from a first end 804 to a second end 806. As used herein, a “manifold” is a structure having two or more openings for making fluid connections. A height of the housing 802 may be less than a width of the housing 802. In some embodiments, the housing 802 may have a generally cuboid shape. However, the housing 802 may take other shapes, as desired. The housing 802 may define a cavity 808 therein. A diaphragm 810 may divide the cavity 808 into a first chamber 808a and a second chamber 808b. In some embodiments, the diaphragm 810 may be preloaded to return to an original configuration after deformation or may be spring loaded. In the illustrated embodiment, the diaphragm 810 is spring loaded. However, in some embodiments, a biasing mechanism, such as a spring, may not be required. The diaphragm 810 may be formed from a flexible or deformable material, such as, but not limited to, silicone, thermoplastic elastomers, flexible metals, and the like.

The housing 802 may include a first port 812, a second port 814, and a third port 816. The ports 812, 814, 816 may be formed as a monolithic structure with the housing 802. However, this is not required. In some embodiments, the ports 812, 814, 816 may be separately formed and subsequently coupled with a respective opening 818, 820, 822 extending through a sidewall of the housing 802. The ports 812, 814, 816 may be generally tubular structures with each port 812, 814, 816 defining a lumen extending therethrough. The lumens of the ports 812, 814, 816 may be configured to selectively fluidly couple the cavity 808 of the housing 802 with another component, such as, but not limited to, a fluid or water supply tube and/or a gas supply tube. In the illustrated embodiment, the first and third ports 812, 816 are positioned adjacent to the second end 806 and the second port 814 is positioned adjacent to the first end 806. However, this is not required. The ports 812, 814, 816 may be positioned in other locations, as desired.

A biasing member 824, such as, but not limited to, a spring, may be positioned within the second chamber 808b. The biasing member 824 may extend between an inner surface of the housing 802 and a surface of the diaphragm 810. The biasing member 824 may be configured to bias the diaphragm 810 towards the first chamber 808a of the housing 802 in the absence of an external biasing force. In some embodiments, the biasing member 824 may be positioned within the first chamber 808a.

The system 800 may further include a gas supply tubing 826 coupled to the first port 812. The gas supply tubing 826 may extend from a first end coupled to the first port 812 to a second end (not explicitly shown) configured to be coupled to the gas/lens wash connection 290 on the outside of the connector portion 265. The gas supply tubing 826 may define a lumen 828 configured to supply air or an alternative gas (e.g., from the air pump 215 or an alternative gas source) to the first chamber 808a of the housing 802.

A fluid supply tubing 830 may be coupled to the second port 814. The fluid supply tubing 830 may extend from a first end coupled to the second port 814 to a second end (not explicitly shown) configured to be coupled to a fluid supply source. In some embodiments, the fluid supply tubing 830 may be configured to be coupled to one of the ports 308a, 308b of the first fluid reservoir 302 of the system 300 of FIG. 3. For example, the fluid supply tubing 830 may include a spike port adaptor (not explicitly shown) configured to be received within one of the ports 308a, 308b. Alternatively, in the system 300 of FIG. 3, the second outlet leg 354 of the branched connector 350 may include a spike port, similar in form and function to the ports 308a, 308b described herein, configured to receive a spike port adaptor of the fluid supply tubing 830. The fluid supply tubing 830 may define a lumen 832 configured to supply water or fluid to the second chamber 808b of the housing 802. A one-way valve 834, or other flow control mechanism, may be positioned in-line with the lumen 832 of the fluid supply tubing 830. In some examples, the one-way valve 834 may be positioned within the second opening 820 of the housing 802. In other examples, the one-way valve 834 may be positioned within the second port 814 or within the fluid supply tubing 830. The one-way valve 834 may be configured to allow a flow of fluid into the second chamber 808b, as shown at arrow 836, while preventing fluid from exiting the second chamber 808b via the second port 814.

A lens wash supply tubing 838 may be coupled to the third port 816. The lens wash supply tubing 838 may extend from a first end coupled to the third port 816 to a second end (not explicitly shown) configured to be coupled to the gas/lens wash connection 290 on the outside of the connector portion 265 to supply lens wash fluid to the endoscope 100. The lens wash supply tubing 838 may define a lumen 840 configured to supply water or fluid from the second chamber 808b of the housing 802 to the endoscope 100. A one-way valve 842, or other flow control mechanism, may be positioned in-line with the lumen 840 of the lens wash supply tubing 838. In some examples, the one-way valve 842 may be positioned within the third opening 822 of the housing 802. In other examples, the one-way valve 842 may be positioned within the third port 816 or within the lens wash supply tubing 838. The one-way valve 842 may be configured to allow a flow of fluid from the second chamber 808b, as shown at arrow 844, while preventing fluid from re-entering the second chamber 808b via the third port 816.

When lens wash is desired, the clinician may press the gas/water valve 140 downward to a second position. Gas is blocked from exiting the valve, allowing pressure of the air passing from the air pump 215 (or alternative gas source, if so provided) to flow through the gas supply tubing 826 and into the first chamber 808a, as shown at arrow 846. As the air is supplied to the first chamber 808a, the pressure inside the first chamber 808a increases. Once the pressure inside the first chamber 808a is sufficient to overcome the biasing force of the biasing member 824, the biasing member 824 is compressed and the diaphragm 810 is moved towards the second chamber 808b of the housing 802, as shown at arrow 848 (FIG. 10B). The movement of the diaphragm 810 may displace the water or fluid within the second chamber 808b causing the water to exit the second chamber 808b via the third port 816, as shown at arrow 844, and to be supplied as lens wash fluid. When the gas/water valve 140 is released, air/gas may exit the first chamber 808a via the first port 812. As the air/gas exits the first chamber 808a, the biasing member 824 moves the diaphragm 810 towards the first chamber 808a of the housing 802. As the diaphragm 810 is moved towards the first chamber 808a of the housing 802, water or other fluid (e.g., from the first fluid reservoir 302) is sucked or pulled into the second chamber 808b via the second port 814, as shown at arrow 836. The diaphragm 810 may fluidly isolate the first chamber 808a from the second chamber 808b.

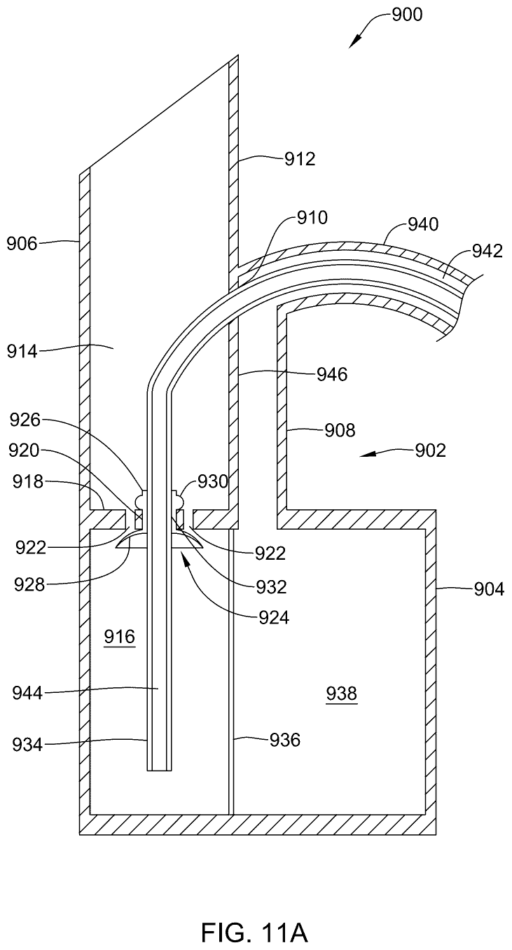

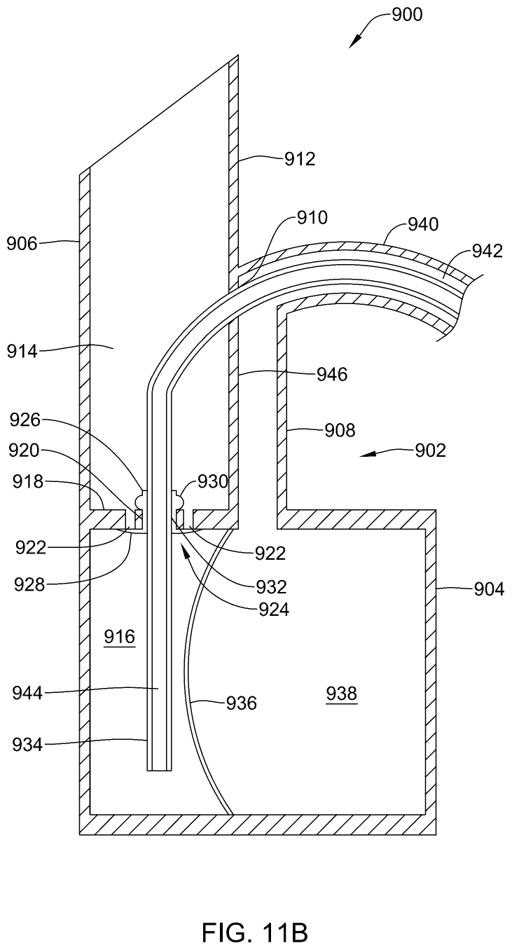

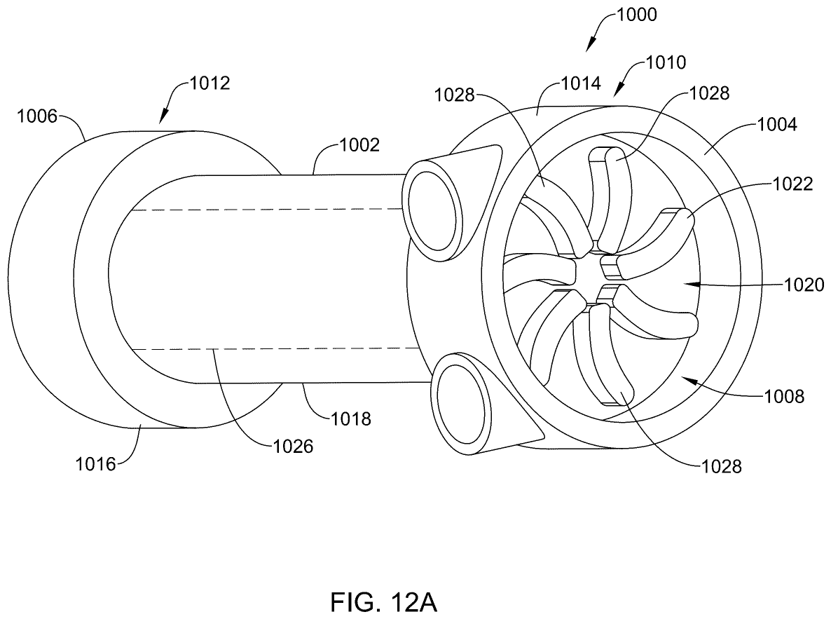

FIG. 11A is a cross-sectional view of another illustrative pressure vessel system 900 in a first configuration that may be used in place of the secondary fluid reservoir 330 of FIG. 3. FIG. 11B is a cross-sectional view of the illustrative system 900 of FIG. 10A in a second configuration. The pressure vessel system 900 may be configured to be fluidly and/or mechanically coupled to a fluid source, such as the first fluid reservoir 302 of FIG. 3.

The pressure vessel system 900 may include a manifold assembly 902 including a manifold housing 904. As used herein, a “manifold” is a structure having two or more openings for making fluid connections. The manifold housing 904 may include a first fluid inlet 906, a second fluid inlet 908, and a first fluid outlet 910. The first fluid inlet 906 may include a spike port adaptor 912. The spike port adaptor 912 may be received within the port of a fluid source. For example, the spike port adaptor 912 may be inserted directly into one of the ports 308a, 308b of the first fluid reservoir 302 of the system 300 of FIG. 3. The spike port adaptor 912 may define a lumen 914 configured to selectively fluidly couple the first fluid reservoir 302, or other fluid source, with a first chamber 916 of the manifold housing 604.

A partition 918 may extend across a cross-section of the spike port adaptor 912 to separate the lumen 914 of the spike port adaptor 912 from the first chamber 916. The partition 918 may include a central aperture 920 extending through a thickness thereof. One or more additional apertures 922 may be radially spaced from the central aperture 920. The one or more apertures 922 may be configured to allow fluid to flow from the lumen 914 of the spike port adaptor 912 into the first chamber 916. In some embodiments, the apertures 922 may be uniformly spaced about the central aperture 920. In other examples, the apertures 922 may be eccentrically spaced about the central aperture 920. A flow control member 924 may extend through the central aperture 920. The flow control member 924 may be configured to selectively allow a flow of fluid from the lumen 914 of the spike port adaptor 912 into the first chamber 916. In some embodiments, the flow control member 924 may be a one-way valve. In the illustrated embodiment, the flow control member 924 may be an umbrella valve, although other one-way valves may be used, as desired. The flow control member 924 may be configured to selectively allow fluid to flow into the first chamber 916 while preventing fluid from flowing from the first chamber 916 into the spike port adaptor 912.

The flow control member 924 may include a central post 926 and an annular flap 928. A lumen 932 may extend from a first end to a second end of the central stem or post 926. The post 926 may be configured to extend through the central aperture 920 in the partition 918. The post 926 may further include an enlarged region 930 extending about a circumference thereof. The enlarged region 930 may be larger than a diameter of the central aperture 920 to prevent the flow control member 924 from disengaging from the central aperture 920. The flow control member 924 may be coupled to the manifold housing 904 using a number of techniques including, but not limited to, glue, adhesives, sonic welding, ultrasound welding, etc. In some cases, the central post 926 of the flow control member 924 may extend through the central lumen 920 to secure the flow control member 924 to the manifold housing 904, for example, by a snap fit or friction fit. The central post 926 may be secured through the central lumen 920 so as to provide a fluid-tight seal between the flow control member 924 and the walls of the central lumen 920. The fluid-tight seal may prevent fluid from entering and/or exiting the first chamber 916 via the central aperture 920 extending through the partition 918.

A lumen 932 may extend from a first end to a second end of the central stem or post 926. The lumen 932 may be sized and shaped to receive a lens wash supply tube 934 therethrough. In some embodiments, the lens wash supply tube 934 may be formed as a single monolithic structure with the first fluid outlet 910. In other embodiments, the lens wash supply tube 934 and the first fluid outlet 910 may be formed as separate components that are subsequently coupled together. The outer surface of the lens wash supply tube 934 may form a fluid-tight seal with the lumen 932 of the central post 926. The fluid-tight seal may prevent fluid from entering and/or exiting the first chamber 916 via the lumen 932 of the flow control member 924.

The annular flap 928 may be movable between a curved configuration (FIG. 11A) in which an outer edge of the flap 928 curves away from the partition 918 and a generally planar configuration (FIG. 11B) in which the flap 928 is generally positioned against a surface of the partition 918. In the curved configuration, the flap 928 may be spaced a distance from the apertures 922 to allow a flow of fluid through the apertures 922. When the first chamber 916 is full or when the pressure inside the first chamber 916 is increased, the flap 928 may be biased against the apertures 922 to prevent fluid from flowing from the spike port adaptor 912 into the first chamber 916 and to prevent fluid from flowing from the first chamber 916 into the spike port adaptor 912. When the fluid level or pressure within the first chamber 916 drops, the flap 928 may return to the curved configuration such that fluid can flow into the first chamber 916.

A diaphragm 936 may fluidly isolate the first chamber 916 of manifold housing 904 from a second chamber 938 of the manifold housing 904. The diaphragm 936 may be formed from a flexible or deformable material that can be stretched and subsequently return to an original configuration. For example, in response to an increase in air volume in the second chamber 938, the diaphragm 936 may expand into the first chamber 916 reducing the volume of the first chamber 916. The diaphragm 936 may take many forms.

The second fluid inlet 908 may be configured to be coupled to a gas supply tubing 940. The gas supply tubing 940 may extend from a first end coupled to the second fluid inlet 908 to a second end (not explicitly shown) configured to be coupled to the gas/lens wash connection 290 on the outside of the connector portion 265. In some embodiments, the gas supply tubing 940 may be formed as a single monolithic structure with the second fluid inlet 908. In other examples, gas supply tubing 940 and the second fluid inlet 908 may be formed as separate components that are subsequently coupled together in a fluid-tight manner. The gas supply tubing 940 may define a lumen 942 configured to supply air or an alternative gas (e.g., from the air pump 215 or an alternative gas source) to the second chamber 938 of the manifold housing 904.

As described above, a lens wash supply tubing 934 may be in fluid communication with and/or coupled to the first fluid outlet 910. The lens wash supply tubing 934 may extend from a first end disposed within the first chamber 916 to a second end (not explicitly shown) configured to be coupled to the gas/lens wash connection 290 on the outside of the connector portion 265 to supply lens wash fluid to the endoscope 100. The lens wash supply tubing 934 may define a lumen 944 configured to supply water or fluid from the first chamber of the manifold housing 704 to the endoscope 100.

In some embodiments at least a portion of the gas and lens wash supply tubing 940, 934 may be coaxially arranged. For example, the gas supply tubing 940 may define a lumen that is sufficiently large in diameter to encompass a smaller diameter lens wash tubing 934, coaxially received within the gas supply tubing 940, as well as provide air to the water source in an annular space surrounding the lens wash tubing 934 to pressurize the second chamber 938. The lens wash supply tubing 934 may be configured to exit the lumen 942 defined by the coaxial gas supply tubing 940 in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265 and/or at the manifold housing 904. In other embodiments, the gas and lens wash supply tubing 940, 934 may be arranged in a side-by-side arrangement.

In the illustrated embodiments, the first fluid inlet 910 extends through a shared sidewall 946 of the first and second fluid inlets 906, 908 such that the lens wash supply tubing 934 extends through the lumen 942 of the gas supply tubing 940, through the first fluid inlet 910, into the lumen 914 of the spike port adaptor 912, and into the first chamber 916. However, it is contemplated that the lens wash supply tubing 934 may be arranged in other configurations, as desired.

When lens wash is desired, the clinician may press the gas/water valve 140 downward to a second position. Gas is blocked from exiting the valve, allowing the pressure of the air passing from the air pump 215 (or alternative gas source, if so provided) to flow through the gas supply tubing 940 and into the second chamber 938 via the second fluid inlet 908. The pressure of the gas may increase the volume of the second chamber 938 and move the diaphragm 936 towards the first chamber 916 (see, for example, FIG. 11B). The pressure of the first chamber 916 may be increased as the volume thereof is decreased. Pressurizing the first chamber 916 forces water out of the lens wash supply tubing 934, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. When the gas/water valve 140 is released, air/gas may exit the second chamber 938 and the diaphragm 936 may return to its original configuration creating a negative pressure in the first chamber 916. This may cause the flow control member 924 to return to a curved open configuration once again allowing fluid to enter the first chamber 916 from the lumen 914 of the first fluid inlet 906.

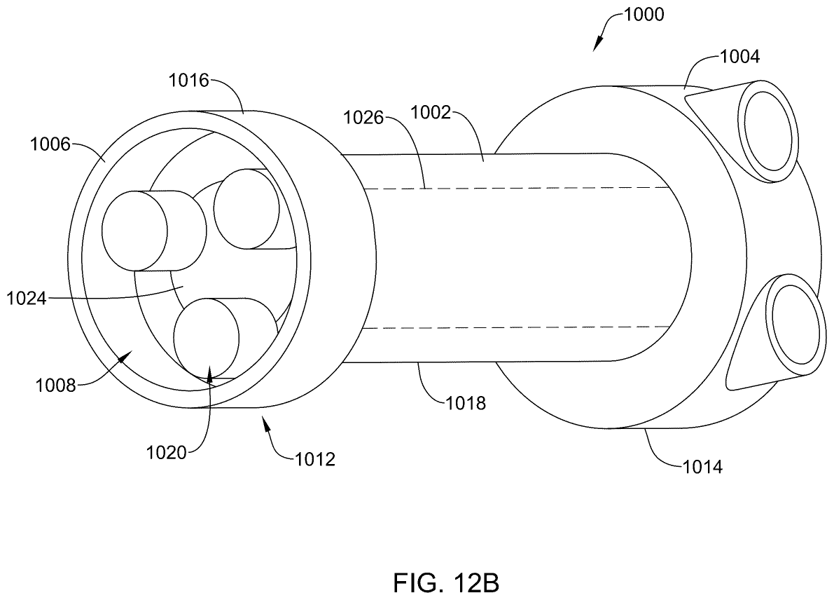

FIG. 12A is a perspective view of a first end of an illustrative drive system 1000 for generating torque to supply a lens wash fluid. FIG. 12B is a perspective view of a second end of the system 1000 of FIG. 12A. Generally, the system 1000 may use air/gas to drive spin a drive wheel generating torque which drives a pump or metering system to supply lens wash fluid.

The system 1000 may include a tubular housing 1002 extending from a first end 1004 to a second end 1006. A lumen 1008 may extend from the first end 1004 to the second end 1006. In some cases, the inner and/or outer diameter of the housing 1002 may vary from the first end 1004 to the second end 1006. In some instances, the housing 1002 may include a first end region 1010 proximate to the first end 1004 and a second end region 1012 proximate to the second end 1006. In some embodiments, the first end region 1010 and the second end region 1012 may include shoulders or enlarged regions, such as flanges 1014, 1016 positioned adjacent to the first end 1004 and the second end 1006 of the housing 1002. The flanges 1014, 1016 may be sized and shaped to house portions of an impeller and drive unit assembly. An intermediate region 1018 may extend between the flanges 1014, 1016.

In some embodiments, the first flange 1014 may have a first outer diameter and the second flange 1016 may have a second outer diameter. The outer diameter of the first flange 1014 and/or the second flange 1016 may be greater than the outer diameter of the intermediate region 1018. The inner diameter of the first flange 1014 and/or the second flange 1016 may be greater than the inner diameter of the intermediate region or saddle 1018. In some instances, the first and second outer diameters may be approximately the same, while in other instances, the first and second outer diameters may be different. In some instances, the first and second inner diameters may be approximately the same, while in other instances, the first and second inner diameters may be different.

A turbine and drive unit assembly 1020 may be rotatably disposed within the lumen 1008 of the housing 1002. The turbine and drive unit assembly 1020 may include a turbine 1022, a drive unit 1024, and an elongate shaft 1026 extending between the turbine 1022 and the drive unit 1024. In some embodiments, the turbine 1022, the drive unit 1024, and/or the elongate shaft 1026 may be formed as separate components that are coupled together. In other embodiments, two or more of the turbine 1022, the drive unit 1024, and/or the elongate shaft 1026 may be formed as a single monolithic structure. The turbine 1022 may include a plurality of vanes 1028. As will be described in more detail herein, a flow of air/gas may be captured by the vanes 1028 to rotate the turbine and drive unit assembly 1020.

The first flange 1014 may include a first air/gas inlet 1030 and a first air/gas outlet 1032. A gas supply tubing (not explicitly shown) may be fluidly coupled to the air/gas inlet 1030. The gas supply tubing may define a lumen configured to supply air or an alternative gas (e.g., from the air pump 215 or an alternative gas source) to an interior of the first flange 1014. In some embodiments, the first flange 1014 may include an end wall configured to enclose the turbine 1022 within the housing 1002. A backpressure valve (not explicitly shown) may be mounted to the air/gas outlet 1032. The backpressure valve may be configured to vent air/gas from the gas supply tubing to atmosphere.

When lens wash is desired, the clinician may press the gas/water valve 140 downward to a second position. Gas is blocked from exiting the valve, allowing pressure of the air passing from the air pump 215 (or alternative gas source, if so provided) to flow through the gas supply tubing and into first flange 1014 via the air/gas inlet 1030. The pressure in the first flange 1014 may increase and open the backpressure valve mounted on the air/gas outlet 1032. As the gas flow into the chamber of the first flange 1014, the gas may contact or push the vanes 1028 to drive the turbine and drive unit assembly 1020 in rotational manner. The gas may flow around the interior of the first flange 1014 in a circular manner with the turbine and drive unit assembly 1020 and vent to atmosphere via the air/gas outlet 1032.

The rotation of the turbine and drive unit assembly 1020 may also rotate the drive unit 1024 to generate torque to drive a pump. The drive unit 1024 may be operationally coupled to a pump configured to supply fluid or water via a lens wash supply tube to the endoscope 100. The pump of the drive system 1000 could be any number of pumping or metering configurations including centrifugal pumps (turbocharger/water pump), positive displacement pumps (peristaltic, piston/screw type), and the like. The pump may be assembled with the drive unit 1024 in the procedure room or may be provided as an assembly with the system 1000. It is contemplated that the ratio of the turbine 1022 to the drive unit 1024 may be tuned or varied to create a desired torque or flow differential. Alternatively, or additionally, a gearing transmission may be provided between the turbine 1022 and the drive unit 1024 to alter the speed or torque ratios of the turbine 1022 to drive unit 1024 sides of the system 1000. When the gas/water valve 140 is released, the flow of air/gas to the interior of the first flange 1014 is stopped and the pressure within the interior of the first flange 1014 drops. When the pressure drops below the cracking pressure of the backpressure valve, air flow is prevented from spinning the turbine and drive unit assembly 1020 (and thus from driving the pump) and also air from the atmosphere is prevented from reversing flow into the endoscope 100.

While not explicitly shown, in some embodiments, the turbine and drive unit assembly 1020 may be replaced by a wound clock spring. For example, at the beginning of the procedure or before the first procedure of the day, a clinician may wind the clock spring until taut. The clock spring mechanism may be held in place by a ratcheting keeper. The ratcheting keeper may only allow the spring drive mechanism to spin in the winding direction. One end of the ratcheting keeper may be connected to a lever arm. This lever arm may then be connected to the gas supply line from the processing unit 210. When the user depresses the lens wash button, the air pump 215 may increase the pressure on the lever arm. The lever arm may release the clock spring keeper allowing the drive side to spin and in turn directly drive the pump side (e.g., the drive unit) to spin and deliver lens wash. When the user releases the gas/water valve 140 the pressure may decrease and the ratcheting keeper may seat back into place stopping the spring drive mechanism from spinning in the unwinding direction and stopping a flow of fluid for lens wash.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A container and tube set arranged and configured to couple to an endoscope for use in an endoscopic procedure, the container and tube set comprising:
a first container configured to contain a fluid, the first container having a first port in fluid communication with a bottom portion thereof;
a pressure vessel system, the pressure vessel system including a manifold housing defining a cavity and including a first fluid inlet in selective fluid communication with the first container, a second fluid inlet, and a first fluid outlet;
a first water supply tube including a first end, a second end, and a first lumen extending therethrough, wherein the first lumen is in selective fluid communication with the cavity of the manifold housing and the second end of the first water supply tube is positioned external to the second container;
a first gas supply tube including a first end, a second end, and a second lumen extending therethrough, wherein the second lumen is in operative fluid communication with the cavity of the manifold housing and the second end of the first gas supply tube is positioned external to the second container; and
a first flow control member positioned within the first fluid inlet.

2. The container and tube set of claim 1, further comprising a second flow control member disposed between the cavity of the manifold housing and the first water supply tube.

3. The container and tube set of claim 1, wherein the first flow control member comprises a floating stopper.

4. The container and tube set of claim 1, wherein the first flow control member comprises a one-way valve.

5. The container and tube set of claim 1, wherein the first flow control member comprises an umbrella valve.

6. The container and tube set of claim 2, wherein the second flow control member comprises a floating stopper.

7. The container and tube set of claim 2, wherein the second flow control member comprises a one-way valve.

8. The container and tube set of claim 2, wherein the second flow control member comprises a linear displacement valve.

9. The container and tube set of claim 1, further comprising a diaphragm disposed within the cavity of the manifold housing, the diaphragm fluidly isolating a first chamber of the cavity from a second chamber of the cavity.

10. The container and tube set of claim 9, wherein the first water supply tube is in fluid communication with the first chamber and the first gas supply tube is in fluid communication with the second chamber.

11. The container and tube set of claim 9, wherein the diaphragm is formed from a flexible material.

12. The container and tube set of claim 1, further comprising a piston disposed within the cavity of the manifold housing, the piston having a seal member disposed around a perimeter thereof and fluidly isolating a first chamber of the cavity from a second chamber of the cavity.

13. The container and tube set of claim 12, further comprising a biasing member disposed between an end of the piston and the first fluid outlet, the biasing member configured to bias the piston towards the second fluid inlet.

14. The container and tube set of claim 1, wherein the first fluid inlet comprises a spike port adaptor.

15. The container and tube set of claim 1, further comprising a second water supply tube including a first end, a second end, and a third lumen extending therethrough, wherein the third lumen is in selective fluid communication with the cavity of the manifold housing via a second fluid outlet and the second end of the second water supply tube is positioned external to the second container.

16. A container and tube set arranged and configured to couple to an endoscope for use in an endoscopic procedure, the container and tube set comprising:
a first container configured to contain a fluid, the first container having a first port in fluid communication with a bottom portion thereof; and
a pressure vessel system comprising:
a tubing valve including a central lumen extending from a proximal end to a distal end thereof and one or more channels laterally spaced from the central lumen and extending from the proximal end to the distal end of the tubing valve;
a proximal gas supply tubing including a proximal end, a distal end coupled to the proximal end of the tubing valve, and a first lumen extending therethrough, wherein the first lumen is in fluid communication with the one or more channels of the tubing valve;
a proximal water supply tubing including a proximal end, a distal end coupled to the proximal end of the tubing valve, and a second lumen extending therethrough, wherein the second lumen is in selective fluid communication with the central lumen of the tubing valve and the first container;
a distal gas supply tubing including a proximal end coupled to the distal end of the tubing valve, a distal end, and a third lumen extending therethrough, wherein the third lumen is in fluid communication with the one or more channels of the tubing valve; and
a distal water supply tubing including a proximal end coupled to the distal end of the tubing valve, a distal end, and a fourth lumen extending therethrough, wherein the second lumen is in selective fluid communication with the central lumen of the tubing valve.

17. The container and tube set of claim 16, wherein the pressure vessel further comprises a slit valve disposed between the distal end of the proximal water supply tubing and the proximal end of the distal water supply tubing.

18. The container and tube set of claim 16, wherein the pressure vessel further comprises a push button valve assembly disposed in-line with the proximal water supply tube.

19. The container and tube set of claim 16, further comprising a one-way valve disposed in a wall of the proximal water supply tubing.

20. A drive system for generating torque to supply a fluid to an endoscope system, the drive system comprising:
a tubular housing including a first end, a second end and a lumen extending therethrough, the tubular housing including a first air inlet and a first air outlet disposed adjacent to the first end thereof; and
a turbine and drive unit assembly rotatably disposed within the lumen of the tubular housing, the turbine and drive assembly comprising:
a turbine;
a drive unit; and
an elongate shaft extending between the turbine and the drive unit;
wherein rotation of the turbine is configured to rotate the drive unit.
